(19) United States
(12) Patent Application Publication
(10) Pub. No.: US 2024/0423689 A1
(43) Pub. Date: Dec. 26, 2024

(54) SAFETY DEVICES FOR CRYOABLATION PROBE

(71) Applicant: Boston Scientific Scimed, Inc., Maple Grove, MN (US)

(72) Inventors: Kyle True, Minneapolis, MN (US); Timothy A. Ostroot, Cokato, MN (US); Cory Ross Stenberg, St. Michael, MN (US); Benjamin Wai-Man Chan, Edina, MN (US); Katrina Marie Daley, Corcoran, MN (US); David Cory Kirt, Minnetonka, MN (US); Alexander James Wiedmann, Columbia Heights, MN (US); Zachary Nickle, Minneapolis, MN (US); Jessica Bechly, Maple Grove, MN (US); Daniel Ryan, Savage, MN (US)

(21) Appl. No.: 18/671,627

(22) Filed: May 22, 2024

Related U.S. Application Data
(60) Provisional application No. 63/468,966, filed on May 25, 2023.

(51) Int. Cl. A61B 18/02

(52) U.S. Cl. CPC A61B 18/0218; A61B 2018/0231

(57) ABSTRACT

In an embodiment, a method for detecting leaks in a shaft of a cryoablation system includes placing a vacuum pump in fluid communication with a return tube and a supply tube; pulling a vacuum within the supply tube and the return tube; after pulling the vacuum for a predetermined amount of time, measuring a vacuum pressure within the supply tube and the return tube; and analysis of the vacuum pressure to assist with identifying leaks in the system.

### RELATED APPLICATIONS

This application claims the benefit of U.S. Provisional Application No. 63/468,966, filed May 25, 2023, the content of which is herein incorporated by reference in its entirety.

### TECHNICAL FIELD

Embodiments herein relate to cryoablation systems and more particularly to safety devices for cryoablation systems.

### BACKGROUND

During cryosurgery, a surgeon may deploy one or more cryoprobes to ablate a target area of a patient anatomy by freezing and thawing the tissue. In one example, a cryoprobe uses the Joule-Thomson effect to produce cooling or heating of the probe tip. In such cases, the expansion of a cryofluid in the cryoablation probe from a higher pressure to a lower pressure leads to cooling of the device tip to temperatures at or below those corresponding to cryoablation a tissue in the vicinity of the tip. Heat transfer between the expanded cryofluid and the outer walls of the cryoprobe leads to formation of an ice ball, in the tissue around the tip and consequent cryoablation the tissue.

### SUMMARY

In a first aspect, a method for detecting leaks in a shaft of a cryoablation system, where the shaft can include a supply tube, a return tube, and an expansion chamber towards a distal end of the shaft. The shaft can be configured to allow a working fluid to travel from a proximal end to the distal end of the shaft, expand in the expansion chamber, and travel back to the proximal end of the shaft between the supply tube and the return tube. The method can include placing a vacuum pump in fluid communication with the return tube and supply tube, pulling a vacuum within the supply tube and the return tube, after pulling the vacuum for a predetermined amount of time, and measuring a vacuum pressure within the supply tube and the return tube. The method can further include, if the vacuum pressure can be at or below a threshold pressure value, determining that there can be no leaks in the shaft, and, if the vacuum pressure can be above the threshold pressure value, determining that there can be a leak in the shaft.

In a second aspect, in addition to one or more of the preceding or following aspects, or in the alternative to some aspects, further can include upon determining that there can be a leak in the shaft, recording a leak indication.

In a third aspect, in addition to one or more of the preceding or following aspects, or in the alternative to some aspects, the threshold pressure can be approximately 0.05 Torr (6.67 Pa).

In a fourth aspect, in addition to one or more of the preceding or following aspects, or in the alternative to some aspects, the predetermined amount of time can be between about 10 seconds and about 45 seconds.

In a fifth aspect, in addition to one or more of the preceding or following aspects, or in the alternative to some aspects, the method further can include, upon pulling the vacuum within the supply tube and the return tube, continuously monitoring the vacuum pressure over time during the predetermined time.

In a sixth aspect, in addition to one or more of the preceding or following aspects, or in the alternative to some aspects, the method further can include, upon measuring a continuous increase in the vacuum pressure towards ambient pressure, determining that the shaft can be not closed at the distal end.

In a seventh aspect, in addition to one or more of the preceding or following aspects, or in the alternative to some aspects, the method further can include: monitoring an initial rise in the vacuum pressure, after the initial rise in vacuum pressure, monitoring that the vacuum pressure can have stabilized to a value that can be above the threshold pressure value, and determining that there can be a leak in the shaft.

In an eighth aspect, in addition to one or more of the preceding or following aspects, or in the alternative to some aspects, the method further can include: monitoring an initial rise in the vacuum pressure, after the initial rise in vacuum pressure, monitoring that the vacuum pressure can have stabilized to a value that can be at or below the threshold pressure value, and determining that there can be no leaks in the shaft.

In a ninth aspect, in addition to one or more of the preceding or following aspects, or in the alternative to some aspects, the method further can include: monitoring an initial rise in the vacuum pressure, measuring a rate of change in the initial rise in the vacuum pressure, and if the rate of change can be above a threshold rate, determining that the shaft can be compromised.

In a tenth aspect, in addition to one or more of the preceding or following aspects, or in the alternative to some aspects, the method can be performed before the cryoablation system is introduced into a patient.

In an eleventh aspect, in addition to one or more of the preceding or following aspects, or in the alternative to some aspects, the method can be performed after the cryoablation system is introduced into a patient, during or after steering the cryoablation system to a treatment site.

In a twelfth aspect, in addition to one or more of the preceding or following aspects, or in the alternative to some aspects, the method can be performed after a first cryoablation procedure is completed with the cryoablation system and before a second cryoablation procedure is performed with the cryoablation system.

In a thirteenth aspect, a cryoablation system can include a working gas circuit, a vacuum chamber, a vacuum pump configured to pull a vacuum on one of the working gas circuit and the vacuum chamber, a console configured to toggle the vacuum pump between the working gas circuit and the vacuum chamber, and a pressure sensor configured to measure a vacuum pressure in the working gas circuit. The console can be further configured to: toggle the vacuum pump to the working gas circuit, control the vacuum pump to pull a vacuum on the working gas circuit, after the vacuum can have been pulled for a predetermined amount of time, measure a vacuum pressure in the working gas circuit with the pressure sensor, and if the vacuum pressure can be above a threshold pressure value, determining that a shaft can be compromised.

In a fourteenth aspect, in addition to one or more of the preceding or following aspects, or in the alternative to some aspects, the console can be further configured to pull the vacuum within the working gas circuit, and continuously monitor the vacuum pressure over time during the predetermined time.

In a fifteenth aspect, in addition to one or more of the preceding or following aspects, or in the alternative to some aspects, the console can be further configured to measure a continuous increase in the vacuum pressure towards ambient pressure and determine that the shaft can be not closed at a distal end.

In a sixteenth aspect, in addition to one or more of the preceding or following aspects, or in the alternative to some aspects, the console can be further configured to: monitor an initial rise in the vacuum pressure, after the initial rise in vacuum pressure, monitor that the vacuum pressure can have stabilized to a value that can be above the threshold pressure value, and determine that there can be a leak in the shaft.

In a seventeenth aspect, a cryoablation system can be included having a pre-cooler fluid circuit, a working fluid circuit, a vacuum circuit, and a shaft. The shaft can include an insulated portion, wherein the vacuum circuit can be defined within the insulated portion, and an expansion chamber. The expansion chamber can include a supply tube having a distal outlet in the expansion chamber, wherein fluid from the working fluid circuit travels through the supply tube and expands in the expansion chamber. The expansion chamber can further include a first layer, wherein the first layer can be configured to contain fluid from the working fluid circuit, and a second layer, wherein the second layer can be configured to increase a radial strength of the expansion chamber, wherein the shaft can have a lower burst strength in the insulated portion at a first location and a higher burst strength in the expansion chamber at a second location.

In an eighteenth aspect, in addition to one or more of the preceding or following aspects, or in the alternative to some aspects, the shaft includes an inner metal tube, and the first layer can be sealed to the inner metal tube at a distal end of the inner metal tube, where the first location can be a seal location between the first layer and the inner metal tube.

In a nineteenth aspect, in addition to one or more of the preceding or following aspects, or in the alternative to some aspects, the first layer can include PET and the second layer can include a polymer or a braided material.

In a twentieth aspect, in addition to one or more of the preceding or following aspects, or in the alternative to some aspects, further can include a burst valve at the first location.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cryoablation system in accordance with various embodiments herein.

FIG. 2 is a schematic view of portions of a cryoablation system in accordance with various embodiments herein.

FIG. 3 is a schematic view of a portion of a cryoablation shaft is shown in accordance with various embodiments herein in accordance with various embodiments herein.

FIG. 4 is a cross sectional view of the shaft of FIG. 3 taken along section 4-4 in FIG. 3 in accordance with various embodiments herein.

FIG. 5 is a cross-sectional view of the shaft of FIG. 3 taken along section 5-5 in FIG. 3 in accordance with various embodiments herein.

FIG. 6 is a schematic view of the biliary system in accordance with various embodiments herein.

FIG. 7 is a schematic view of the growth of an ice ball generated by a cryoablation system in accordance with various embodiments herein.

FIG. 8 is a schematic view of a portion of a cryoablation shaft in accordance with various embodiments herein.

FIG. 9 is a schematic view of a portion of a cryoablation shaft in accordance with various embodiments herein.

FIG. 10 is a schematic view of a portion of a cryoablation shaft in accordance with various embodiments herein.

FIG. 11 is a schematic view of a cryoablation system in accordance with various embodiments herein.

FIG. 12 is a schematic view of a console for a cryoablation system shown in accordance with various embodiments herein.

FIG. 13 is a schematic view of a testing apparatus for performing the shaft integrity test is shown in accordance with various embodiments herein.

FIG. 14 is a flowchart of a method of performing a shaft integrity test shown in accordance with various embodiments herein.

FIG. 15 is an exemplary plot of shaft pressure vs. time for three different shafts is shown in accordance with various embodiments herein.

FIG. 16 is an exemplary plot of shaft pressure vs. time for two different shafts is shown in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Some cryoablation systems may be useful for ablating lesions in the biliary system or other difficult to access portions of the human anatomy. In such cases, the cryoprobes may have to navigate tortuous passageways. Cryoprobes with rigid shafts and sharp (piercing) tips, may not be suitable for such applications. Cryoprobes with flexible shafts are possible where the shaft may be sufficiently flexible to access particular portions of the human anatomy, such as the biliary system, while simultaneously maintaining adequate burst strength and thermal insulation to ensure patient safety.

In the context of the possibility of navigating to anatomical locations provided by the catheter's flexibility comes an increased value to shaft integrity testing. For both flexible and rigid cryoablation probes, shaft integrity testing can ensure that the cryoablation shaft is a closed system (devoid of leaks) prior to pumping the pressurized cryogenic fluids through the shaft. It is undesirable to use positive pressure to test for leaks in a cryoablation system once the shaft has been inserted into the patient because, in the event of a leak, pressurized gas could flow out of the system and into the patient, causing the hazardous situation the integrity check is designed to prevent. In various embodiments, a shaft integrity test utilizes the ability of the shaft to achieve and maintain vacuum between a supply tube and a return tube to determine that the probe is a closed system. If a sufficiently low level of vacuum cannot be reached and maintained by the shaft, a determination that the shaft has a leak will be made.

A shaft integrity test can be conducted after the probe is steered to a treatment location. As a result, the shaft integrity test can identify any damage that might have occurred during the navigation and/or repositioning process. In addition, or alternatively, a shaft integrity test can be conducted between ablations, such as after a first ablation treatment and before a subsequent ablation treatment.

The concepts described herein can be applied in the context of the cryoablation systems described in US Published Patent Application US2021/00045793, titled “Dual Stage Cryocooler,” and US Published Patent Application US2021/00045794, titled “Flexible Cryoprobe,” both filed Aug. 14, 2020, and both incorporated herein by reference in their entireties.

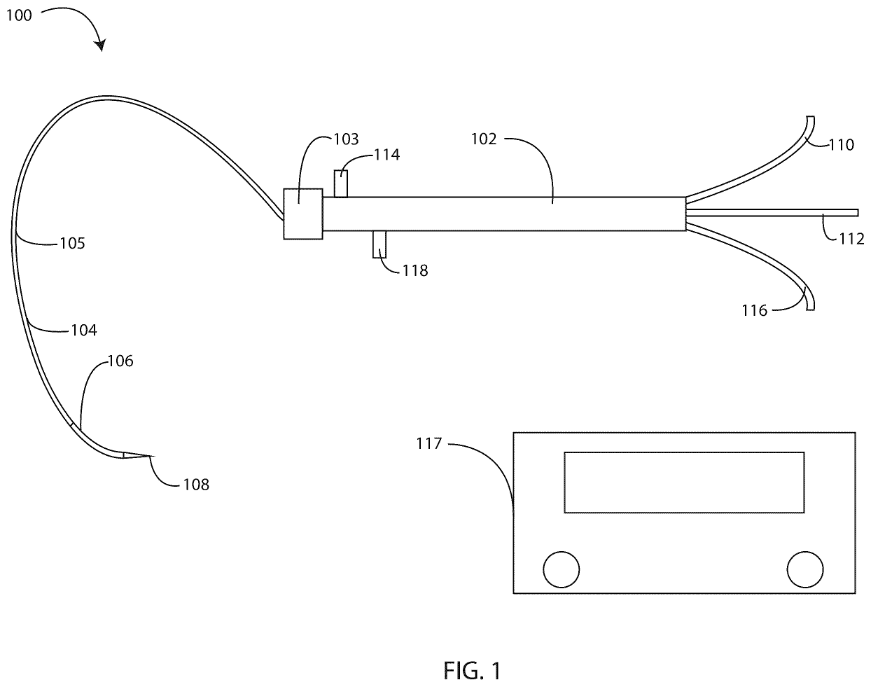

Referring now to FIG. 1, a schematic view of a cryoablation system is shown in accordance with various embodiments herein. In various embodiments, the cryoablation system can include a handle 102 and a shaft 104. In various embodiments, the shaft 104 is insertable into the handle 102 and can be securely attached to the handle with shaft-handle connector 103. In various embodiments, the shaft 104 and the shaft-handle connector 103 of a cryoablation system 100 can form a catheter assembly. In some embodiments, the catheter assembly includes the components of the cryoablation system that are to be replaced each time a cryoablation procedure is performed. In some aspects, the cryoablation system 100 may include a working fluid source 110, a pre-cooler fluid source 112, and vacuum source 114 which are all connectible to the cryoablation system 100.

The three sources correspond to three independent circuits in the cryoablation system 100: pre-cooler, working fluid, and active vacuum. In some embodiments, the working fluid source 110 and pre-cooler fluid source 112 connect to the base of the handle 102 of the cryoablation system 100 and vacuum source 114 connects near the distal end of the handle, adjacent to the shaft-handle connector 103. The cryoablation system may further include a pre-cooler gas exhaust 116 and a working gas exhaust 118 connecting to the handle 102. In various embodiments, the shaft-handle connector 103 functions as a manifold to ensure each of the flow circuits remain isolated from one another.

In some embodiments, the cryoablation system 100 includes a console 117. The console may be used to control the system and may be in electrical and fluid communication with the handle and cryoablation assembly. In some embodiments, the working fluid source 110, pre-cooler fluid source 112, vacuum source 114 may all be connectable to a console 117 of the cryoablation system 100 using conduits. In some embodiments, the pre-cooler gas exhaust 116, working gas exhaust 118, or both can connect to a conduit which carries the exhaust back to the console 117 or other location in the procedure room where the exhaust is vented to the ambient environment at an appropriate location. It should be noted that various sources and exhausts may be placed in position and in any suitable configuration along the handle 102, and that the arrangement of FIG. 1 is just one example of a suitable configuration.

An example of specifications and functions of each of these circuits is provided in the following paragraph. However, it should be noted that the particular fluids and pressure values are meant for exemplary purposes and other configurations are possible.

In an embodiment, the pre-cooler circuit can contain 24.1 mega Pascals (MPa) pressurized Argon. The precooler circuit can cool the incoming stream of working fluid and can operate in the handle. In an embodiment, the working fluid circuit can contain 12.4 MPa pressurized Argon and/or 12.4 MPa pressurized Helium. The working fluid circuit generates and/or thaws ice balls. The working fluid circuit can operate in the handle, the insulated portion or insulated zone of the shaft, and the expansion chamber of the shaft. In an embodiment, the active vacuum can hold a vacuum of less than or equal to 6.67 Pascals (Pa). The active vacuum can insulate the shaft. The active vacuum can operate in the handle and the insulated zone of the shaft.

In various embodiments, the working fluid circuit runs through both the handle 102 and the shaft 104 of the cryoablation system 100 and carries the fluid which both generates and thaws the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. During the ablation (freeze cycle), 12.4 MPa argon is circulated through the probe to generate the ice ball in the patient's body surrounding the expansion chamber 106. The working fluid can be any suitable cooling fluid (e.g., nitrogen, air, argon, krypton, xenon, N2O, CO2, CF4). In some embodiments, the pressure of the high-pressure stream of the working fluid can be greater than or equal to 6.9 MPa, 8.3 MPa, 9.7 MPa, 11.0 MPa, 12.4 MPa, 17.2 MPa, 27.6 MPa, or 41.4 MPa. In some embodiments, the pressure of the high-pressure stream of the working fluid can be less than or equal to 55.2 MPa, 34.5 MPa, 20.7 MPa, 18.6 MPa, 16.5 MPa, 14.5 MPa, or 12.4 MPa. In some embodiments, the pressure of the high-pressure stream of the working fluid can fall within a range of 6.9 MPa to 41.4 MPa, or 8.3 MPa to 27.6 MPa, or 9.7 MPa to 16.5 MPa, or 11.0 MPa to 14.5 MPa, or can be about 12.4 MPa. Accordingly, in the embodiments where the working fluid is a cooling fluid, the temperature of the working fluid at the expansion chamber 106 can be about 190 Kelvin. In some embodiments, the temperature of the working fluid can be less than or equal to 250 Kelvin, 200 Kelvin, 150 Kelvin, or 100 Kelvin, or can be an amount falling within a range between any of the foregoing.

In various embodiments, the pre-cooler circuit is fully contained within the handle 102. In various embodiments, the pre-cooler circuit is located in a console 117 of the system. In various embodiments, the pre-cooler circuit is located in a part of the catheter just proximal to the handle. In various embodiments, the pre-cooler circuit is located in a part of the catheter just distal to the handle. The pre-cooler circuit operates using argon or any other suitable cooling fluid in various embodiments. In some embodiments, the high-pressure stream of the pre-cooler fluid may be at a pressure greater than the pressure of the high-pressure stream of the working fluid. The pre-cooler fluid may, for instance, be supplied at pressures greater than about 13.8 MPa. In some embodiments, the pressure of the pre-cooler fluid can be greater than or equal to 10.3 MPa, 13.8 MPa, 17.2 MPa, 20.7 MPa, or 24.1 MPa. In some embodiments, the pressure of the pre-cooler fluid can be less than or equal to 31.0 MPa, 29.3 MPa, 25.9 MPa, or 24.1 MPa. In some embodiments, the pressure of the pre-cooler fluid can fall within a range of 10.3 MPa to 31.0 MPa, or 13.8 MPa to 29.3 MPa, or 17.2 MPa to 27.6 MPa, or 20.7 MPa to 25.9 MPa, or can be about 24.1 MPa.

In some embodiments, the outer surface of the shaft 104 may be thermally insulated from the inner surface of the shaft. In various embodiments, the vacuum circuit or vacuum chamber runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft. In alternative embodiments, shaft insulation can be obtained by containing a non-circulating fluid or gas within an insulating shaft.

The shaft 104 can be of any suitable length capable of reaching the target anatomy in the subject. In some embodiments, the shaft length can be greater than or equal to 20 cm, 38 cm, 55 cm, 72 cm, or 90 cm. In some embodiments, the shaft length can be less than or equal to 150 cm, 135 cm, 120 cm, 105 cm, or 90 cm. In some embodiments, the shaft length can fall within a range of 20 cm to 150 cm, or 38 cm to 135 cm, or 55 cm to 120 cm, or 72 cm to 105 cm, or can be about 90 cm.

In various embodiments, certain portions of the shaft 104 may be flexible. In an embodiment, the entire length of the shaft may be flexible. For instance, the shaft may be bendable about its lengthwise axis. In some such embodiments, the shaft may have a shaft diameter configured such that the shaft may be sufficiently flexible to form a curve having a desired radius of curvature. For instance, the shaft may be sufficiently flexible, such that the shaft may form a curve having the smallest radius of curvature of less than or equal to 30 mm, 20 mm, 10 mm, or 5 mm.

In various embodiments, shaft 104 may include an insulated zone 105 and an expansion chamber 106. The insulated zone 105 defines the portion of shaft 104 that is insulated by the vacuum chamber. The expansion chamber 106 defines the portion of the shaft 104 that is not insulated by the vacuum and where the ice ball is generated. In various embodiments, flexible shaft carries high pressure working fluid from the handle 102 to the expansion chamber 106, where it undergoes a Joule-Thompson expansion and corresponding temperature change. The working fluid exits down the flexible shaft, through the handle, before venting to the atmosphere from the console, or into the handle and venting from the handle.

The distal end of the shaft may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

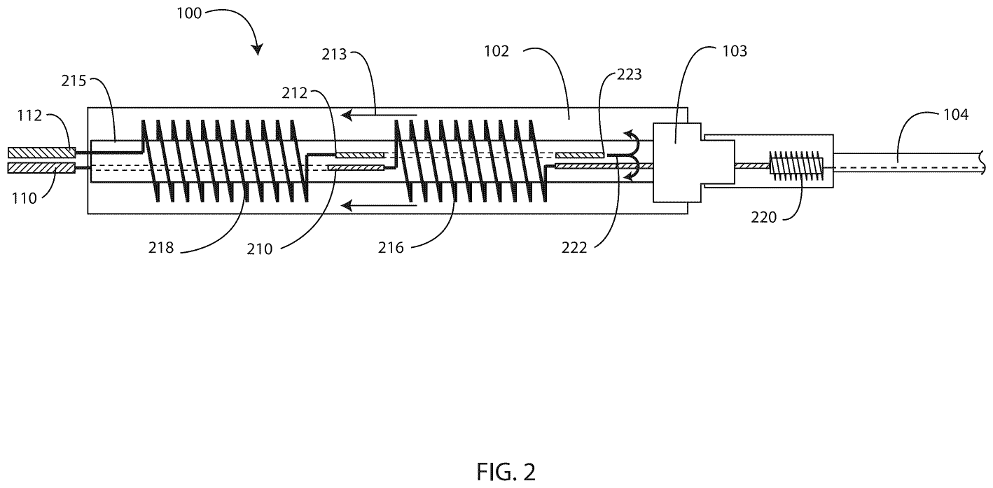

### Handle with Pre-Cooler Circuit (FIG. 2)

Referring now to FIG. 2, a schematic view of portions of a cryoablation system is shown in accordance with various embodiments herein. In some aspects, the cryoablation system 100 may include a working fluid source 110 connecting to a working fluid circuit and a pre-cooler fluid source 112 connecting to a pre-cooler fluid circuit. The working fluid circuit may include a working fluid supply conduit 210 for carrying a high-pressure stream of the working fluid from the working fluid source 110 to the distal end of the shaft 104 (not shown in this view). The working fluid circuit may also include a working fluid return conduit (not shown in this view) for carrying a low-pressure stream of the working fluid from the distal end of the shaft back to the base of the handle 102.

The pre-cooler fluid circuit may include a pre-cooler supply circuit 212, which terminates at pre-cooler Joule-Thomson orifice 223 and carries a high-pressure stream of a pre-cooler fluid from the pre-cooler fluid source 112 to the pre-cooler fluid expansion region 222 in the handle 102. The pre-cooler fluid circuit may also include a pre-cooler return conduit (marked by arrows 213). The pre-cooler return conduit may be configured to carry the pre-cooler fluid away from the pre-cooler fluid expansion region 222 back to the base of the handle 102. The pre-cooler return conduit may be housed along with the pre-cooler supply circuit 212 and extend back to a control console and gas manifold.

In various embodiments, the pre-cooler fluid circuit may facilitate heat exchange between the working fluid and the pre-cooler fluid. For instance, the pre-cooler fluid circuit can be used to precool the high-pressure stream of the working fluid in embodiments where the working fluid cools upon expansion to cryogenically ablate tissue surrounding the distal operating tip 108. In various embodiments, the working fluid supply conduit 210 may include a first heat exchanger 216. The first heat exchanger 216 may facilitate heat exchange between the high-pressure stream of the working fluid in the working fluid supply conduit 210 and the low-pressure stream of the pre-cooler fluid in the pre-cooler return conduit.

In various embodiments, the pre-cooler supply conduit 212 may include a second heat exchanger 218 that permits heat exchange between the high-pressure stream of the pre-cooler fluid and the low-pressure stream of the pre-cooler fluid (e.g., recuperative heat exchange). In various embodiments aspects, the pre-cooler fluid may also be a cooling fluid. In such embodiments, recuperative heat exchange between the high-pressure stream of the pre-cooler fluid and the low-pressure stream of the pre-cooler fluid may remove heat from the high-pressure stream of the pre-cooler fluid. Accordingly, the second heat exchanger 218 may facilitate precooling the high-pressure stream of the pre-cooler fluid.

In various embodiments, the high-pressure stream of the pre-cooler fluid leaving the second heat exchanger 218 continues to flow through the pre-cooler supply conduit 212 to the pre-cooler fluid expansion region 222. In the pre-cooler fluid expansion region, which is fully contained in handle 102, the pre-cooler supply conduit 212 terminates in a Joule-Thomson orifice. The high-pressure stream of the pre-cooler fluid may undergo expansion at or downstream of the Joule-Thomson orifice in the pre-cooler fluid expansion region 222. The rapid drop in pressure causes a corresponding drop in temperature. The pre-cooler fluid expansion region 222 may be in fluid communication with the pre-cooler return conduit to carry the expanded low-pressure stream of the pre-cooler fluid (e.g., to vent to atmosphere, if the pre-cooler fluid circuit is an open circuit, or back to a pre-cooler fluid source if the pre-cooler fluid circuit is a closed circuit). After expansion at the Joule-Thomson orifice, the chilled pre-cooler fluid passes back through handle 102, in the annular space between the core tube 215 and the outer surface of the handle 102. As the pre-cooler fluid passes through the pre-cooler return conduit, it cools the working fluid at the first heat exchanger 216.

The working fluid circuit 210 may also include a third heat exchanger 220 in the shaft 104 of the cryoablation system that is configured for heat exchange (e.g., recuperative heat exchange) between the high-pressure stream of the working fluid in the working fluid supply circuit 210 and the low-pressure stream of the working fluid returning through the shaft 104 (not shown in this view).

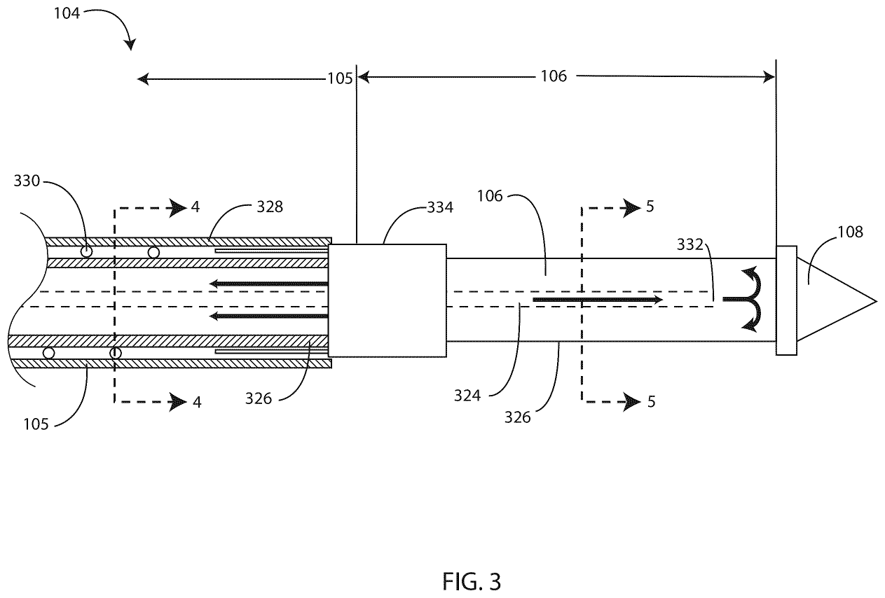

### Distal Tip and Expansion Chamber Details (FIG. 3)

Referring now to FIG. 3, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 which is located within a return tube 326, which is located within an insulating shaft 328. The concentric-shaft construction is designed to isolate the working fluid circuit 210 and vacuum chamber 336 from each other.

In various embodiments, after exiting the handle 102, the high-pressure flow of the working fluid travels down the supply tube 324. When the working fluid reaches the working fluid expansion chamber 106, the supply tube 324 terminates in a Joule-Thomson orifice 332 or distal outlet 332. The high-pressure stream of the working fluid may undergo expansion at or downstream of the Joule-Thomson orifice 332 in expansion chamber 106. The rapid drop in pressure causes a corresponding drop in temperature. Heat transfer between the expanded working and the outer walls of expansion chamber 106 leads to formation of an ice ball in the tissue around the tip 108 resulting in cryoablation of the tissue.

The expansion chamber 106 may be in fluid communication with the working fluid return conduit (defined by the annular space between the supply tube 324 and the inner surface of the return tube 326 of the expansion chamber) to carry the expanded low pressure stream of the working fluid (e.g., to vent to atmosphere, if the working fluid circuit is an open circuit, or back to a working fluid source if the working fluid circuit is a closed circuit). As the working fluid passes through the working fluid return conduit, it cools the working fluid input stream at the third heat exchanger 220 (FIG. 2).

In various embodiments, the working fluid is a cooling fluid and a cooling gas (e.g., nitrogen, air, argon, krypton, xenon, N₂O, CO₂, CF₄). In such cases, the high-pressure stream of the working fluid may be at a pressure such that expansion via the Joule-Thomson orifice 332 may result in the working fluid cooling to temperatures for cryogenically ablating tissue surrounding the expansion chamber 106. In certain aspects, the pressure of the high-pressure stream of the working fluid upstream of the Joule-Thomson orifice 332 can be between about 6.9 MPa and about 13.8 MPa (e.g., about 12.4 MPa). Accordingly, in the embodiments where the working fluid is a cooling fluid, the temperature of the working fluid after expansion from the Joule-Thomson orifice 332 can be greater than or equal to 150, 160, 170, 180, 190, or 200 Kelvin, or can be an amount falling within a range between any of the foregoing.

Cryoablation system 100 can be designed such that the outermost surface of the shaft does not cause thermal damage to non-target structures. In various embodiments, Ice ball formation is limited to the expansion chamber 106 of the shaft 104, which can also be referred to as the active region of the device. Selective ice ball formation is achieved by pulling a vacuum through the insulated zone 105 of shaft 104. In various embodiments, the cryoablation system 100 may be configured for establishing vacuum communication between the shaft 104 and vacuum source 114.

Referring back to FIG. 1, cryoablation system 100 may be configured to connect to a vacuum source 114 at handle 102. In various embodiments, the vacuum source 114 is configured to pull vacuum along the length of the insulated zone 105 of shaft 104. In an embodiment, vacuum is pulled between the outer diameter of the return tube 326 and the inner diameter of the insulating shaft 328 throughout the insulated zone 105 of shaft 104.

In various embodiments, the vacuum source 114 is configured to pull a vacuum within at least a portion of the handle 102. Such a configuration can insulate the handle 102 and protect the cryoablation system operator from cryogenic exhaust gases. In some embodiments the vacuum source 114 is connected to the handle 102 and the shaft 104 is in fluid communication with the handle 102 such that pulling a vacuum in the handle can also evacuate the space between the supply tube 324 and return tube 326. In other embodiments, the vacuum source 114 is connected directly to the shaft 104, for example, with the use of a t-fitting along the length of the shaft 104.

To provide for thermal insulation along the insulated zone 105 of shaft 104, the wall of the flexible shaft is a double wall (a return tube surrounded by an insulating shaft) with a small gap between the return tube 326 and the insulating shaft 328. By pulling a vacuum between the return tube and the insulating shaft, convective heat transfer is prevented, so that the temperature of the working fluid does not ablate or cause uncontrolled apoptosis/necrosis to healthy non-target patient tissue along the insulated zone of the shaft. Adequate thermal insulation is obtained by actively pumping out the air in the gap and maintaining a vacuum of about 0.05 torr. However, other vacuum pressures may be appropriate depending on the configuration of the cryoablation system. In some embodiments, a supporting filament 330 is wrapped around the outer diameter of the return tube 326. One option for the filament material is a polymer such as polyether ether ketone (PEEK). The filament may prevent direct contact between the return tube outer surface and insulating shaft inner surface. Filament 330 minimizes thermal conduction between the inner and insulating shafts. Other alternatives may be used in place of the filament 330 such as an extruded tubing/co-extrusion shape or other features placed onto the shaft.

In some embodiments, the shaft may not include a filament. In such embodiments the return tube 326 and the insulating shaft 328 are selected to have material properties that are sufficient to minimize thermal conduction between the inner shaft and the insulating shaft.

A joint 334 is present at the junction of the insulated zone 105 and the expansion chamber 106. This joint is capable of sealing the vacuum layer.

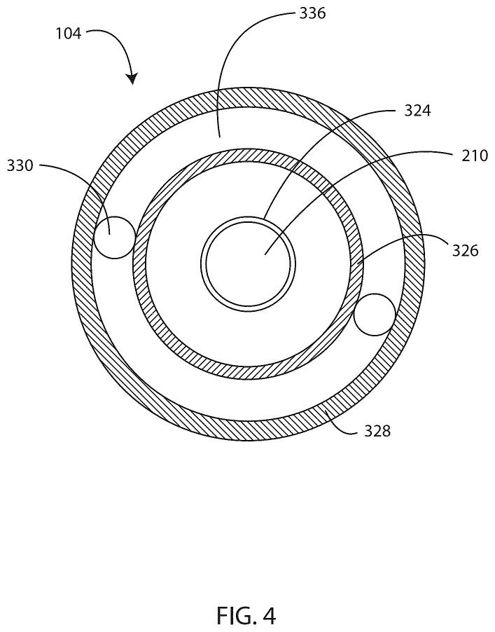

### Flexible Shaft Cross-Section, Dimensions and Materials (FIG. 4)

Referring now to FIG. 4, a cross sectional view of the shaft of FIG. 3 taken along section 4-4 is shown in accordance with various embodiments herein. In various embodiments, the insulated zone 105 of shaft 104 includes a supply tube 324 concentrically located within a return tube 326, which is concentrically located within an insulating shaft 328. The insulated zone 105 can include a shaft portion of the vacuum chamber 336 and an insulated portion of the working gas circuit 210. In various embodiments, the vacuum chamber 336 surrounds and is isolated from the insulated portion of the working gas circuit 210.

In various embodiments, after exiting the handle 102, the high-pressure flow of the working fluid travels distally down the insulated zone of the shaft through supply tube 324. After cooling and expansion in the expansion chamber 106, the working fluid travels proximally back through the insulated zone 105 of the shaft 104 in the annular space between the supply tube 324 and return tube 326.

In various embodiments, the materials and dimensions of each of the layers of the shaft 104 may be selected to provide sufficient degree of flexibility for the shaft to be bendable about its longitudinal axis at the working temperatures of the device.

In various embodiments, the supply tube 324, which also may be referred to as a capillary tube herein, is constructed from any suitable material or materials such as flexible metals, polymers, composites, or the like. In an embodiment, the supply tube 324 is constructed from Nitinol (NiTi), stainless steel, or the like.

In some embodiments, the inner diameter of the supply tube 324 can be greater than or equal to 0.30 mm, 0.35 mm, 0.40 mm, or 0.45 mm. In some embodiments, the inner diameter of the supply tube 324 can be less than or equal to 0.60 mm, 0.55 mm, 0.50 mm, or 0.45 mm. In some embodiments, the diameter of the supply tube 324 can fall within a range of 0.30 mm to 0.60 mm, or 0.35 mm to 0.55 mm, or 0.40 mm to 0.50 mm, or can be about 0.45 mm.

In some embodiments, the outer diameter of the supply tube 324 can be greater than or equal to 0.38 mm, 0.43 mm, 0.48 mm, 0.53 mm, or 0.58 mm. In some embodiments, the outer diameter can be less than or equal to 0.78 mm, 0.73 mm, 0.68 mm, 0.63 mm, or 0.58 mm. In some embodiments, the outer diameter can fall within a range of 0.38 mm to 0.78 mm, or 0.43 mm to 0.73 mm, or 0.48 mm to 0.68 mm, or 0.53 mm to 0.63 mm, or can be about 0.58 mm.

In some embodiments, the thickness of the supply tube 324 can be greater than or equal to 0.10 mm, 0.11 mm, 0.12 mm, 0.14 mm, or 0.15 mm. In some embodiments, the thickness of the supply tube 324 can be less than or equal to 0.20 mm, 0.19 mm, 0.18 mm, 0.16 mm, or 0.15 mm. In some embodiments, the thickness of the supply tube 324 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the return tube 326 is constructed from any suitable material or materials such as flexible metals, polymers, or the like. In various embodiments, the return tube 326 can be made of polyimide, fluorinated ethylene propylene (FEP), Teflon, or the like. In an embodiment, the return tube 326 is formed from a polyimide material as it is highly impermeable to gases at a wide range of temperatures and can thus contain the working fluid inside and hold vacuum on the outside. In a particular example, the return tube 326 is made of a braid-reinforced polyimide tube to enhance gas impermeability, burst strength, and flexibility. In some embodiments, the return tube 326 is formed from a single layer of material. In some embodiments, the return tube 326 can be formed from two or more layers of material selected to optimize the performance of the shaft 104. The layers of material can be bonded together using any suitable technique or techniques such as adhesives, reflow processes, or the like.

In some embodiments, the outer diameter of the return tube 326 can be greater than or equal to 1 mm, 1.1 mm, 1.2 mm, 1.3 mm, or 1.4 mm. In some embodiments, the outer diameter of the return tube 326 can be less than or equal to 1.8 mm, 1.7 mm, 1.6 mm, 1.5 mm, or 1.4 mm. In some embodiments, the outer diameter of the return tube 326 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the inner diameter of the return tube 326 can be greater than or equal to 0.9 mm, 1.0 mm, 1.1 mm, 1.2 mm, or 1.3 mm. In some embodiments, the inner diameter of the return tube 326 can be less than or equal to 1.7 mm, 1.6 mm, 1.5 mm, 1.4 mm, or 1.3 mm. In some embodiments, the inner diameter of the return tube 326 can fall within a range of 0.9 mm to 1.7 mm, or 1.0 mm to 1.6 mm, or 1.1 mm to 1.5 mm, or 1.2 mm to 1.4 mm, or can be about 1.3 mm.

In some embodiments, the thickness of the return tube 326 can be greater than or equal to 0.10 mm, 0.11 mm, 0.12 mm, 0.14 mm, or 0.15 mm. In some embodiments, the thickness of the return tube 326 can be less than or equal to 0.20 mm, 0.19 mm, 0.18 mm, 0.16 mm, or 0.15 mm. In some embodiments, the thickness of the return tube 326 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the insulating shaft 328 is constructed from any suitable material or materials such as flexible metals, polymers, or the like. In various embodiments, the insulating shaft 328 is made of polyimide, fluorinated ethylene propylene (FEP), Teflon, or the like. In a particular embodiment, the insulating shaft 328 may include polytetrafluoroethylene (PTFE), and/or one or more polyether block amides (known under the tradename Pebax®, hereinafter “Pebax”).

In some embodiments, the insulating shaft 328 is formed from a single layer of material. In some embodiments, the insulating shaft 328 can be formed from two or more layers of material selected to optimize the performance of the shaft 104. The layers of material can be bonded together using any suitable technique or techniques such as adhesives, reflow processes, or the like.

In an embodiment, the insulating shaft may be formed using a braid-reinforced polyimide tube skim-coated with a Pebax outer layer. Such a tri-layer construction enables the deep vacuum to be maintained between the return tube and the insulating shaft without causing the insulating shaft 328 to collapse onto the return tube 326.

In some embodiments, the outer diameter of the insulating shaft 328 can be greater than or equal to 1.2 mm, 1.4 mm, 1.5 mm, 1.6 mm, or 1.8 mm. In some embodiments, the outer diameter of the insulating shaft can be less than or equal to 2.2 mm, 2.1 mm, 2.0 mm, 1.9 mm, or 1.8 mm. In some embodiments, the outer diameter of the insulating shaft can fall within a range of 1.3 mm to 2.3 mm, or 1.4 mm to 2.1 mm, or 1.5 mm to 2.0 mm, or 1.6 mm to 1.9 mm, or can be about 1.8 mm.

In some embodiments, the inner diameter of the insulating shaft 328 can be greater than or equal to 1.0 mm, 1.2 mm, 1.3 mm, 1.4 mm, or 1.6 mm. In some embodiments, the inner diameter of the insulating shaft 328 can be less than or equal to 2.2 mm, 2.0 mm, 1.9 mm, 1.8 mm, or 1.6 mm. In some embodiments, the inner diameter of the insulating shaft 328 can fall within a range of 1.0 mm to 2.2 mm, or 1.2 mm to 2.0 mm, or 1.3 mm to 1.9 mm, or 1.4 mm to 1.8 mm, or can be about 1.6 mm.

In some embodiments, the thickness of the insulating shaft 328 can be greater than or equal to 0.10 mm, 0.11 mm, 0.12 mm, 0.14 mm, or 0.15 mm. In some embodiments, the thickness of the insulating shaft 328 can be less than or equal to 0.20 mm, 0.19 mm, 0.18 mm, 0.16 mm, or 0.15 mm. In some embodiments, the thickness of the insulating shaft 328 can fall within a range of 0.10 mm to 0.20 mm, or 0.11 mm to 0.19 mm, or 0.12 mm to 0.18 mm, or 0.14 mm to 0.16 mm, or can be about 0.15 mm.

In some embodiments, a PEEK filament 330 is wound around the return tube 326. PEEK filament 330 may have pitch that is greater than or equal to 0.5 mm, 1.0 mm, 1.5 mm, or 2.0 mm, or can be an amount falling within a range between any of the foregoing. Alternatively, the filament may be a plurality of discrete pieces attached along the return tube 326. The PEEK filament 330 prevents direct contact between the return tube 326 and the insulating shaft 328, maintaining their coaxial alignment. In some embodiments, an adhesive (e.g., Loctite) is applied on the filament at the end of the return tube 326 and the insulating shaft 328 to attach the PEEK filament 330. In various embodiments, the PEEK filament wrap is configured to minimize or prevent conductive heat transfer from the return tube to the insulating shaft. In alternative embodiments, other insulating polymers may be used as a substitute for the PEEK filament such as expanded PTFE (ePTFE), nylon, or the like.

In some embodiments, the diameter of the PEEK filament 330 can be greater than or equal to 0.002 mm, 0.004 mm, or 0.005 mm. In some embodiments, the diameter of the PEEK filament 330 can be less than or equal to 0.007 mm, 0.006 mm, or 0.005 mm.

In some embodiments, the diameter of the PEEK filament 330 can fall within a range of 0.002 mm to 0.007 mm, or 0.004 mm to 0.006 mm, or can be about 0.005 mm.

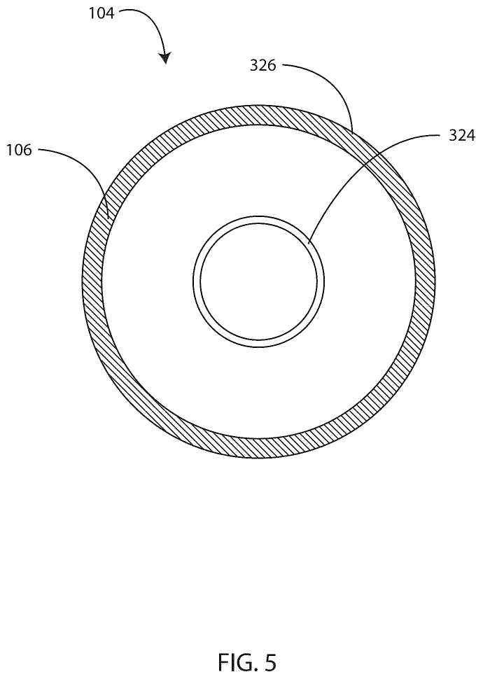

### Shaft in the Expansion Chamber (FIG. 5)

Referring now to FIG. 5, a cross-sectional view of the shaft of FIG. 3 taken along section 5-5 is shown in accordance with various embodiments herein. The cross-sectional view of FIG. 5 depicts expansion chamber 106 of the shaft 104. In various embodiments, the expansion chamber 106 is distal to the insulated zone 105 along the shaft 104. The expansion chamber 106 can include an expansion portion of the working fluid circuit 210.

In various embodiments, after exiting the handle 102, the high-pressure flow of the working fluid travels down the supply tube 324. After cooling and expansion in expansion chamber 106, the working fluid travels back down through the expansion chamber 106 in the annular space between the supply tube 324 and the outer wall of the expansion chamber. In various embodiments, the expansion chamber 106 is configured to maximize the heat transfer between the working gas and the patient's tissue through the optimization of parameters such as wall thickness, materials, and the like.

In various embodiments, the expansion chamber 106 is constructed from any suitable material or materials such as flexible metals, polymers, or the like. In various embodiments, the expansion chamber 106 is made of polyimide, fluorinated ethylene propylene (FEP), Teflon, or the like. In some embodiments, the expansion chamber 106 includes a continuation of the return tube 326 of the insulated zone 105 of the shaft 104. Alternatively, the expansion chamber is a separate component from the return tube 326 that can be joined to the flexible shaft 104 using any suitable joint and/or fitting, such as reflow processes, glue joints, solder joints, or any other suitable mechanical joining process capable of withstanding cryogenic pressures and temperatures.

In some embodiments, the expansion chamber 106 is formed from a single layer of material. In some embodiments, the expansion chamber 106 is formed from two or more layers of material. The layers of material can be bonded together using any suitable technique or techniques such as adhesives, reflow processes, or the like.

In some embodiments, the outer diameter of the expansion chamber 106 can be greater than or equal to 1.3 mm, 1.4 mm, 1.5 mm, 1.6 mm, or 1.7 mm. In some embodiments, the outer diameter of the expansion chamber 106 can be less than or equal to 2.1 mm, 2.0 mm, 1.9 mm, 1.8 mm, or 1.7 mm. In some embodiments, the outer diameter of the expansion chamber 106 can fall within a range of 1.3 mm to 2.1 mm, or 1.4 mm to 2.0 mm, or 1.5 mm to 1.9 mm, or 1.6 mm to 1.8 mm, or can be about 1.7 mm.

In some embodiments, the inner diameter of the expansion chamber 106 can be greater than or equal to 1.0 mm, 1.1 mm, 1.2 mm, 1.3 mm, or 1.4 mm. In some embodiments, the inner diameter of the expansion chamber 106 can be less than or equal to 1.8 mm, 1.7 mm, 1.6 mm, 1.5 mm, or 1.4 mm. In some embodiments, the inner diameter of the expansion chamber 106 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the thickness of the wall of expansion chamber 106 can be greater than or equal to 0.20 mm, 0.22 mm, 0.25 mm, 0.28 mm, or 0.30 mm. In some embodiments, the thickness of the wall of the expansion chamber 106 can be less than or equal to 0.40 mm, 0.38 mm, 0.35 mm, 0.32 mm, or 0.30 mm. In some embodiments, the thickness of the wall of the expansion chamber 106 can fall within a range of 0.20 mm to 0.40 mm, or 0.22 mm to 0.38 mm, or 0.25 mm to 0.35 mm, or 0.28 mm to 0.32 mm, or can be about 0.30 mm.

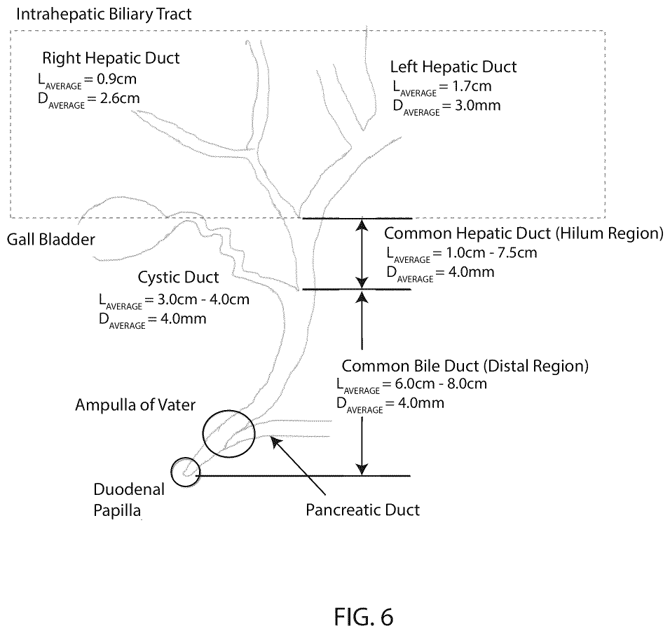

### Treatment Area Examples (FIG. 6)

The cryoablation systems and structures described herein are flexible and well-suited to cryoablate an area along a lumen, vessel, or passageway of the body. The present cryoablation system 100 is configured to be adequately flexible to access and ablate many different such structures. The expansion chamber may be shaped and sized to generate an ice ball or ice cylinder of appropriate geometry for ablating structures along the length of a body lumen. Moreover, the materials and configuration of the probes described herein are selected to protect patient tissue and withstand high operating pressures and low temperatures.

Referring now to FIG. 6, a schematic view of the biliary system is shown in accordance with various embodiments herein. The biliary system includes organs and ducts that make and store bile (a fluid made by the liver that helps digest fat) and release it into the small intestine. The biliary system includes the gallbladder and bile ducts inside and outside the liver.

Bile duct cancer or cholangiocarcinoma is a rare disease in which cancer cells form in the bile ducts. Treatment outcomes for cholangiocarcinoma are generally poor. Current treatment options such as a Whipple procedure or biliary drains are high risk and often ineffective.

Cryoablation is a promising treatment for cholangiocarcinoma. The present cryoablation system 100 is configured to be adequately flexible to access and ablate the bile ducts of a patient. The expansion chamber may be shaped and sized to generate an ice ball of appropriate geometry for ablating tumors in the bile ducts.

In addition to the treatment of cholangiocarcinoma, the cryoablation system 100 can be used to treat a number of conditions including other cancerous tumors (e.g., skin, liver, kidney, bone, lung, prostate and breast), pain, skin conditions (e.g., atypical moles, warts, skin tags or actinic keratosis), arrythmia, or the like. The cryoablation system 100 can additionally ablate benign masses, soft tissue, and healthy tissue.

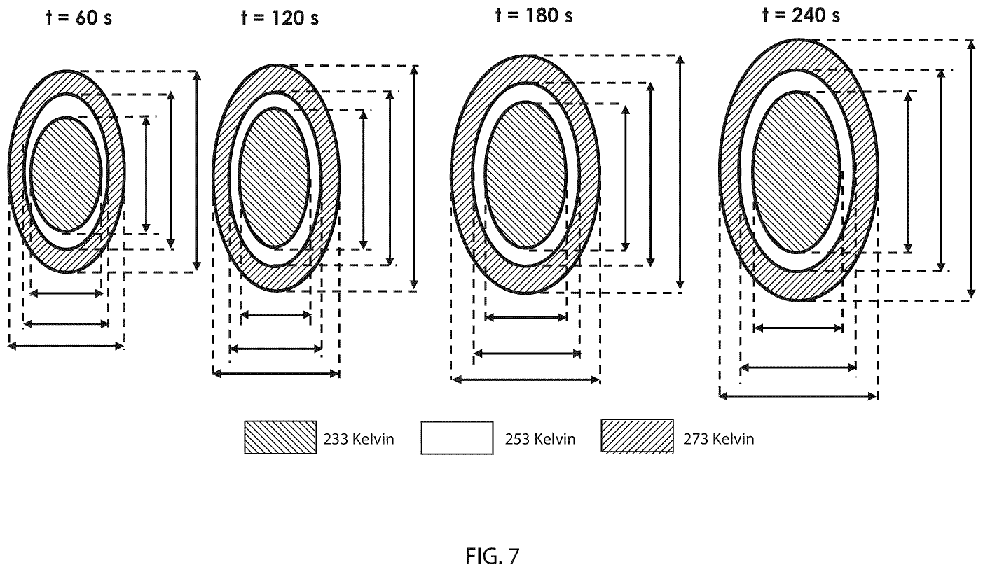

### Ice Ball Formation (FIG. 7)

Referring now to FIG. 7, a schematic view of the growth of an ice ball generated by a cryoablation system is shown in accordance with various embodiments herein. Cryoablation is defined as cell destruction using cold temperatures. An ice ball is formed at the expansion chamber of a cryoablation system, which freezes intra cellular and extracellular material to temperatures colder than 173 Kelvin. The application of these extremely cold temperatures causes cell death. In order to cause complete cell death, sufficiently low ablation temperatures must be achieved. Lethal temperatures for various tissues have been reported between 253 Kelvin and 233 Kelvin.

FIG. 7 illustrates the growth of an ice ball over the course of a cryoablation procedure. The time points in this example are at 60 seconds, 120 seconds, 180 seconds, and 240 seconds. Isotherms for 273 Kelvin, 253 Kelvin and 233 Kelvin are depicted for each period of time. As the cryoablation procedure progresses in time, the overall ice ball size increases. More critically, the cell killing isotherms (the 253 Kelvin and 233 Kelvin isotherms) grow along both the major and minor axes of the ice ball.

It should be noted that the times and temperatures given by FIG. 7 are for exemplary purposes only. Ice ball formation can vary based on the configuration of the cryoablation system and the patient's tissue. Moreover, ice ball growth can stabilize after a certain ablation time has been reached. For example, in some embodiments, an ice ball can reach its maximum diameter after ablating the tissue for approximately 10 minutes. In some embodiments, the cryoablation system operator can terminate a cryoablation procedure upon the ice ball reaching its maximum size. In some embodiments, the cryoablation system operator can continue the cryoablation procedure upon the ice ball reaching its maximum size for a predetermined amount of time, as holding the tissue at cryogenic temperatures for longer times may have therapeutic benefits.

In some embodiments, the ice ball is produced and then thawed, and then produced again. In some embodiments, active thawing techniques are used while in other embodiments passive thawing is used.

In the example of FIG. 7, the ice balls are oblong in shape (e.g., they are longer along a primary axis than a secondary axis). The primary axis of the ice ball corresponds to a lengthwise axis of the expansion chamber. The ratio of expansion chamber height to diameter can be correlated to ice ball shape. In particular, an expansion chamber that is much longer than it is wide will result in a more oblong ice ball. Such an ice ball is generally more suitable for the treatment of cholangiocarcinoma, as it is more compatible with the anatomy of the bile ducts. However, other cryoablation applications may indicate a more spherical ice ball. In such scenarios, the expansion camber can be modified to have a smaller length to diameter ratio.

In some embodiments, the major axis length of an ice ball can be greater than or equal to 0.5 mm, 2 mm, 4 mm, 6 mm, 8 mm, or 10 mm, or can be an amount falling within a range between any of the foregoing. In some embodiments, the minor axis length of an ice ball can be greater than or equal to 0.5 mm, 2 mm, 4 mm, 6 mm, 8 mm, or 10 mm, or can be an amount falling within a range between any of the foregoing.

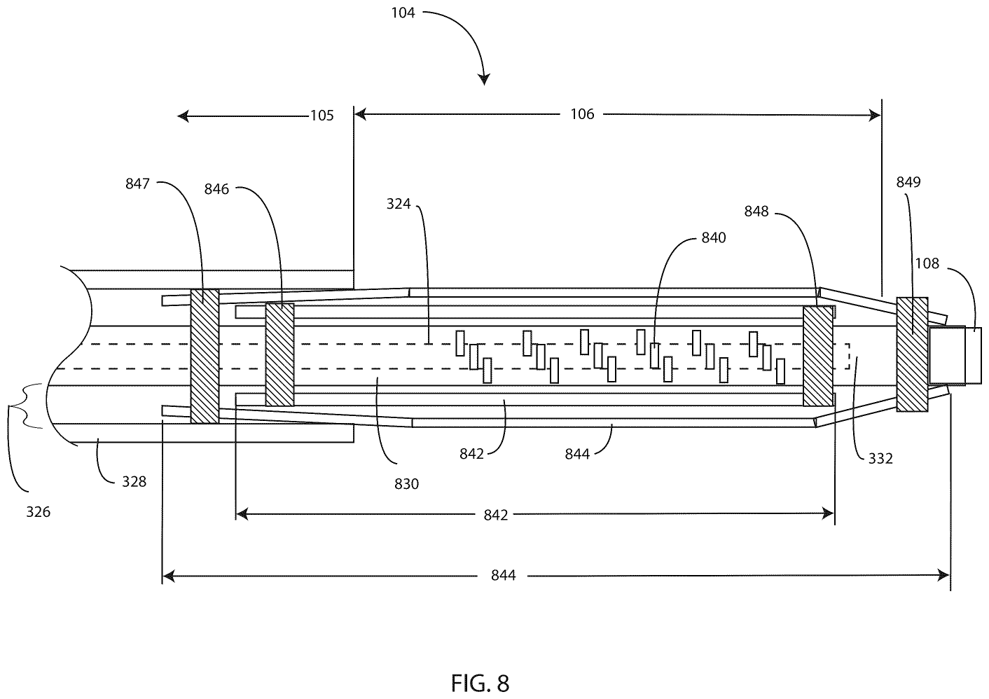

### Cryoablation Shaft including a Slotted Tube Portion, a Polymer Layer, and a Reinforcing Layer (FIG. 8)

Referring now to FIG. 8, a schematic view of a tip portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 concentrically located within a return tube 326, which is concentrically located within an insulating shaft 328. The concentric-shaft construction is designed to isolate the working gas and active vacuum circuits from each other.

In various embodiments, after exiting the handle 102, the high-pressure flow of the working fluid travels down the supply tube 324. The high-pressure stream of the working fluid may undergo expansion at or downstream of the Joule-Thomson orifice 332 and returns down the shaft in the annular space between the supply tube 324 and the return tube 326.

In various embodiments, the expansion chamber 106 of shaft 104 includes a supply tube 324 concentrically located within the return tube 326. The return tube 326 includes multiple layers in various embodiments.

In various embodiments, the innermost layer of the return tube is a slotted tube 830 which includes slots 840 along at least a portion of the length of shaft 104. Slots 840 are formed in the tube material by any suitable means, such as laser cutting. The slots 840 can be laser cut into the shaft using any suitable pattern to optimize the strength and flexibility of the return tube 326. The material of the slotted tube 830 can be metal such as stainless steel, nitinol, or other durable materials. Many different configurations and patterns of slotted tube 830 are available and one can be selected with the flexibility desirable in this application. The slotted tube forms the core of the return tube 326 in various embodiments.

In some embodiments, the slotted tube 830 includes the slots only in the expansion chamber 106. In this embodiment, the portion of the slotted tube 830 that extends through the insulated zone 105 has solid walls. FIG. 8 illustrates this embodiment by showing a schematic side view of the slotted tube 830, including slots 840 in the expansion chamber 106, and showing the supply tube 324 in dashed lines within the slotted tube 830. In an alternative embodiment, the slotted tube 830 has slots along its entire length.

The slotted tube may be configured to be sufficiently flexible to form a curve having a desired radius of curvature. For instance, the slotted tube may be sufficiently flexible, such that the shaft may form a curve having the smallest radius of curvature of less than or equal to 30 mm, 20 mm, 10 mm, 5 mm, or 3 mm. The slotted tube 830 may have different levels of flexibility in the expansion chamber and the insulated zone 105.

In various embodiments, a first polymer layer 842 surrounds the portion of the slotted tube 830 which includes the slots, in order to contain the working fluid within the shaft 104. In various embodiments, a reinforcing layer 844 surrounds the first polymer layer 842 and is configured to provide additional strength and reinforcement to the first polymer layer 842, thus reducing the likelihood of any leak or rupture in the first polymer layer.

In various embodiments, the first polymer layer 842 can be formed from any suitable polymer such as polyethylene terephthalate (PET), PTFE, ePTFE, PEEK, polyetherimide (PEI), polyimide (PI), or the like. In various embodiments, the reinforcing layer 844 can be a second polymer layer formed from any suitable polymer such as PET, PTFE, PEEK, polyetherimide (PEI), polyimide (PI), or the like. In various embodiments, the reinforcing layer is gas impermeable. In various embodiments, the reinforcing layer 844 is not impermeable and can include a braided polymer material and/or a coiled polymer or metallic material and/or coatings & encapsulants.

In various embodiments, the return tube may include two, three, four, or more polymer layers. In various embodiments, the return tube may include two, three, four, or more total layers.

In the embodiment of FIG. 8, where the slotted portion of the slotted tube 830 is within the expansion chamber, the first polymer layer 842 and the reinforcing layer 844 extend from just within the insulated zone 105 to the distal end. In various embodiments, the first polymer layer 842 is bonded to the slotted tube 830 at first bond 846 near the proximal end of the expansion chamber 106 and at a second bond 848 near the tip 108 of the shaft 104.

In various embodiments, the reinforcing layer 844 is bonded to the slotted tube 830 at first bond 847 near the proximal end of the expansion chamber 106 and/or at a second bond 849 near the tip 108 of the shaft 104.

Additional bonds can be placed along any other suitable location of the shaft. The bonds can be formed from any suitable material or materials such as Vectran, UHMWPE, PEEK, Polyimide, Metallic Wire. Vectran is a manufactured fiber, spun from a liquid-crystal polymer that displays increased tensile strength at cold temperatures, making it advantageous for use in cryoablation systems. In alternative embodiments, the bonds 846, 847, 848 and 849 may be formed from alternate techniques such as crimp or swage rings, polymer reflow joints, adhesive joints, solder joints or the like.

In various embodiments, the wrap materials of the bonds 846, 847, 848 and 849, can also be wrapped along the entire length of the expansion chamber around the polymer layer, the reinforcing layer, or both. The wrap layer may have a higher pitch of wraps at the bond location and extend along the remainder of the expansion chamber at a lower pitch level. The wrap may extend from the proximal end to the distal end and then reverse direction and extend back in a proximal direction. The number of wrap layers may be one, two, three, four or more.

In various embodiments, the bonds 846, 847, 848 and 849 are configured to increase the burst strength of the layers of the return tube 326.

In some embodiments, the burst strength of each of the first polymer layer 842 and the reinforcing layer 844 can be greater than or equal to 12.4 MPa, 13.1 MPa, 13.8 MPa, 14.5 MPa, 15.2 MPa, or 41.4 MPa. In some embodiments, the burst strength of each of the first polymer layer 842 and the reinforcing layer can be less than or equal to 41.4 MPa, 20.7 MPa, 19.3 MPa, 17.9 MPa, 16.5 MPa, or 15.2 MPa. In some embodiments, the burst strength can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of the bonded first polymer layer 842 and the reinforcing layer 844 together can be greater than or equal to 12.4 MPa, 13.1 MPa, 13.8 MPa, 14.5 MPa, 15.2 MPa 27.6 MPa, or 41.4 MPa. In some embodiments, the burst strength of the bonded first polymer layer 842 and the reinforcing layer 844 together can be less than or equal to 41.4 MPa, 20.7 MPa, 19.3 MPa, 17.9 MPa, 16.5 MPa, or 15.2 MPa. In some embodiments, the burst strength of the bonded first polymer layer 842 and the reinforcing layer 844 together can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of each of the bonds 846, 847, 848 and 849 can be greater than or equal to 1.4 MPa, 4.8 MPa, 8.3 MPa, 11.7 MPa, 15.2 MPa, 27.6 MPa, or 41.4 MPa. In some embodiments, the burst strength of each of the bonds can be less than or equal to 41.4 MPa, 34.8 MPa, 28.3 MPa, 21.7 MPa, or 15.2 MPa. In some embodiments, the burst strength of each of the bonds can fall within a range of 1.4 MPa to 41.4 MPa, or 4.8 MPa to 34.8 MPa, or 8.3 MPa to 28.3 MPa, or 11.7 MPa to 21.7 MPa, or can be about 15.2 MPa.

In some embodiments, all of the bonds 846, 847, 848 and 849 can have the same burst strength. Alternatively, some of the bonds can have a higher burst strength than others. For instance, proximal bonds 846, 847 may have a lower burst strength than distal bonds 848, 849.

In various embodiments, the return tube 326 may be configured to be sufficiently flexible to form a curve having a desired radius of curvature. For instance, the return tube may be sufficiently flexible, such that the shaft may form a curve having the smallest radius of curvature of less than or equal to 30 mm, 20 mm, 10 mm, 5 mm, or 3 mm.

### Slotted Tube Portion Can Extend the Entire Length of the Return Tube

FIG. 8 illustrates that the slotted tube 830 includes the slots only in the expansion chamber 106, but in an alternative embodiment, the slotted tube 830 has slots along its entire length. In this alternative embodiment, the first polymer layer 842 and the reinforcing layer 844 will also extend along the entire length of the return tube 326. The reinforcing wrap layer, such as a wrap of a Vectran material, may also extend along the entire return tube length. The presence of slots along the entire return tube length may be desirable to achieve the desired level of flexibility along the return tube.

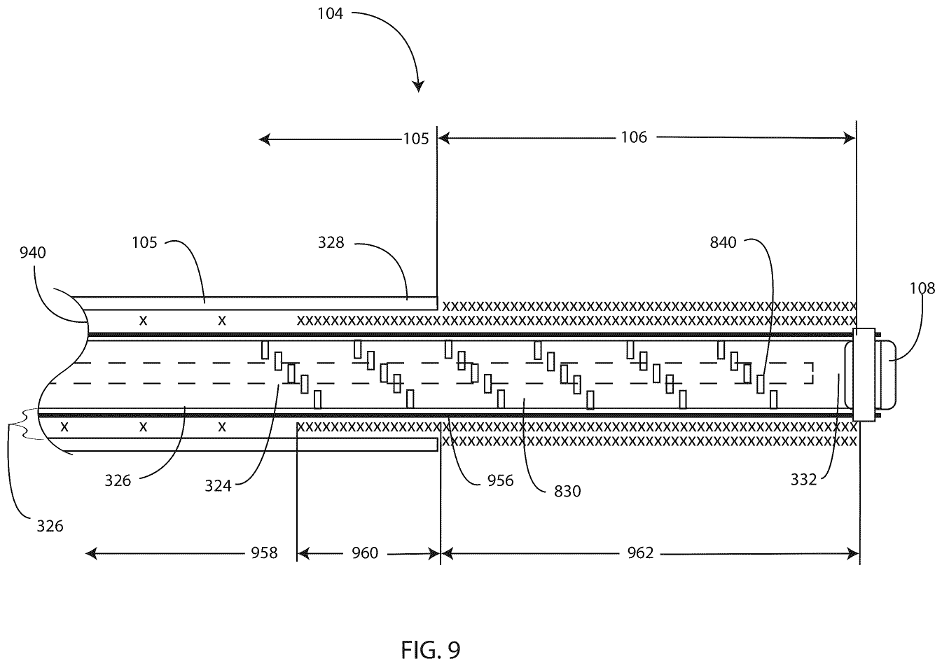

### Cryoablation Shaft including a Slotted Tube Portion and a Gradient Braid (FIG. 9)

Referring now to FIG. 9, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 located within a return tube 326. The return tube 326 of FIG. 9 includes multiple layers.

The innermost layer of the return tube of 326 is a slotted tube 830, which can be constructed using the options and details described herein. The slotted tube 830 is surrounded by a polymer layer 956, which is concentrically surrounded by a reinforcing gradient braid layer 940.

In various embodiments, gradient braid layer 940 includes zones of different density of braid, increasing in density toward the distal end of the device. A first braid zone 958 is present in the insulated zone and is the least dense. A second braid zone 960 overlaps the insulated zone 105 and the expansion chamber 106 and is more dense than the first braid zone 958. The third braid zone 962 is the densest and is present in the expansion zone. The return tube may include one, two, three, or more braid layers.

In various embodiments, the polymer layer 956 is configured to contain the working fluid in the return tube and not allow the working fluid to escape radially through the slots in the slotted tube. The polymer layer may be constructed from the options and materials discussed herein with respect to the first polymer layer herein. In some embodiments, the expansion chamber 106 may include an additional braided layer between the slotted tube 830 and the polymer layer 956 (not shown in this view). The additional braided layer is configured to prevent friction between the polymer layer 956 and the slotted tube 830.

### Braid Materials, Braid Element Diameters, Braid Density Zones, Coils, and Burst Strength Examples

Braided tubes are used in a variety of medical applications. Braid reinforced tubing can improve functional properties such as strength, stiffness, burst pressure resistance, torque transmission, and kink resistance of a medical device. Such features enable can enable a cryoablation shaft to navigate tortuous portions of a patient's anatomy, such as the biliary ducts. Design considerations such as braid pattern, pick count (ppi), material, wire dimension, wire size/shape and durometer of plastics can have significant impact on device performance.

The braided portions of the shaft can be formed from any suitable material or materials such as metals (e.g., nitinol, stainless steel, tungsten, MP35N, or other such materials), polymers, (e.g., PET, Kevlar, Carbon fiber, Vectran, or other such materials), or the like. Braided materials are formed by weaving a metal or fiber filament in a braid pattern. In various embodiments, the cross section of the filament is circular, however other cross-sectional shapes are possible (e.g., flat, star, triangle). In some embodiments, the diameter of the filament can be greater than or equal to 0.01 mm, 0.1 mm, 0.2 mm, 0.3 mm, or 0.5 mm. In some embodiments, the diameter of the filament can be less than or equal to 2 mm, 1.6 mm, 1.2 mm, 0.8 mm, or 0.5 mm. In some embodiments, the diameter of the filament can fall within a range of 0.01 mm to 2.00 mm, or 0.1 mm to 1.6 mm, or 0.2 mm to 1.2 mm, or 0.3 mm to 0.8 mm, or can be about 0.5 mm.

In various embodiments, the density of the braided portions can be altered with more dense braids offering more radial strength and rigidity and less dense braids offering more flexibility. The shaft can include multiple braids having varying material properties. In various embodiments, the shaft may include a light braid (e.g., have relatively low braid density) between the return tube 326 and the polymer layer 956. As described above, the light braid may offer limited structural support but can prevent undue friction between the slotted return tube 326 and the polymer layer 956.

In various embodiments, the density of the braided materials can increase from the base of the shaft 104 to the tip 108 of the shaft. In the event that there is a device failure, such a configuration improves the likelihood of the shaft failing closer to the base. Such a failure mode is generally preferable compared to a failure closer to the tip 108 of the shaft in terms of patient safety outcomes.

In various embodiments, the first gradient braid zone 958 spans the insulated zone 105 of the shaft 104 (starting from where the shaft connects to the handle 102 and terminating at or before the expansion chamber 106). In various embodiments, the gradient braid 958 portion can have a first burst strength. The first burst strength can be constant along the insulated zone of the shaft. Alternatively, the first burst strength of the gradient braid portion 958 can increase from the base of the shaft to the expansion chamber. In various embodiments, the gradient braid portion 958 can have a first braid density. The first braid density can be constant along the insulated zone of the shaft. Alternatively, the first braid density of the gradient braid portion 958 can increase from the base of the shaft to the expansion chamber.

In some embodiments, the minimum burst strength of the gradient braid portion 958 can be less than or equal to 20.7 MPa, 13.8 MPa, 6.9 MPa, 5.5 MPa, 4.1 MPa, 2.8 MPa, 1.4 MPa, or 0.7 MPa, or can be an amount falling within a range between any of the foregoing. In some embodiments, the maximum burst strength of the gradient braid portion 958 can be greater than or equal to 0.7 MPa, 2.0 MPa, 3.4 MPa, 4.8 MPa, 6.2 MPa, 6.9 MPa, 13.8 MPa, or 20.7 MPa or can be an amount falling within a range between any of the foregoing.

In various embodiments, the second braid portion 960 spans the expansion chamber 106 of the shaft 104. In some embodiments, the second braid portion 960 may start at the beginning of the expansion chamber and terminate at or near the tip 108 of the shaft. Alternatively, the second braid portion 960 may start towards the distal end of the insulated zone 105 of the shaft (as depicted by FIG. 9) and terminate at or near the tip 108 of the shaft. In some embodiments, the second braid portion 960 is a continuation of the first braid portion 958. Alternatively, the second braid portion 960 may be made of elements that are physically separate from the elements of first braid zone 958.

In various embodiments, the second braid portion 960 can have a second burst strength. The second burst strength can be constant along the length of the first braid layer. Alternatively, the second burst strength of the second braid portion 960 can increase from the along the length of the first braid layer (from the end of the insulated zone 105 to the tip 108). In various embodiments, the second braid portion 960 can have a second braid density. The second braid density can be constant along the length of the second braid portion. Alternatively, the second braid density of the second braid portion 960 can increase along the length of the second braid portion.

In some embodiments, the burst strength of the second braid portion 960 can be greater than or equal to 10.3 MPa, 12.9 MPa, 15.5 MPa, 18.1 MPa, or 20.7 MPa, or 41.4 MPa. In some embodiments, the burst strength of the second braid portion 960 can be less than or equal to 34.5 MPa, 31.0 MPa, 27.6 MPa, 24.1 MPa, or 20.7 MPa. In some embodiments, the burst strength of the second braid portion 960 can fall within a range of 10.3 MPa to 34.5 MPa, or 12.4 MPa to 31.0 MPa, or 15.5 MPa to 27.6 MPa, or 17.9 MPa to 34.5 MPa, or can be about 20.7 MPa.

In various embodiments, the third braid zone 962 spans the expansion chamber 106 of the shaft 104. In some embodiments, the third braid zone 962 may start at the beginning of the expansion chamber and terminate at or near the tip 108 of the shaft.

In various embodiments, the third braid zone 962 can have a third burst strength. The third burst strength can be constant along the length of the third braid zone. Alternatively, the burst strength of the third braid zone 962 can increase from the along the length of the third braid zone. In various embodiments, the third braid zone 962 can have a third braid density. The braid density can be constant along the length of the third braid zone. Alternatively, the braid density of the third braid zone 962 can increase along the length of the first braid layer.

In some embodiments, the burst strength of the third braid zone 962 can be greater than or equal to 10.3 MPa, 12.9 MPa, 15.5 MPa, 18.1 MPa, or 20.7 MPa. In some embodiments, the burst strength of the third braid zone 962 can be less than or equal to 34.5 MPa, 31.0 MPa, 27.6 MPa, 24.1 MPa, or 20.7 MPa. In some embodiments, the burst strength of the third braid zone 962 can fall within a range of 10.3 MPa to 34.5 MPa, or 12.4 MPa to 31.0 MPa, or 15.5 MPa to 27.6 MPa, or 17.9 MPa to 24.1 MPa, or can be about 20.7 MPa.

In alternative embodiments, any, or all of the gradient braid zones 958, 960, 962 can be formed from coils. A coil, as defined herein is a filament of material wound around the shaft. Like in braided materials, as detailed above, the filament can be selected to have suitable material(s) and cross-sectional shapes including round, rectangular, or other shapes. The zones can include coils of different density, radial strength, rigidity, and flexibility. The coils can be single layered or multi layered. In some embodiments, the multi-layer coils can have alternate wind directions (e.g., first layer is wound clockwise, and the second layer is wound counterclockwise). In various embodiments, the pitch of the coil can be varied to optimize the properties of the shaft, such as flexibility, burst strength, or the like. For instance, tighter pitches of coil can increase the burst strength of the shaft while looser pitches of coil can increase the flexibility of the shaft. In various embodiments, the pitch of the coil materials can increase from the base of the shaft 104 to the tip 108 of the shaft.

### Transitions Between Braid Zones

In some embodiments, the transitions between the first, second and third braid portions are transitions in density of the same physical braid elements, so that one braid zone is a continuation of a neighboring braid zone. Alternatively, one braid portion may be made of elements that are physically separate from the elements of a neighboring braid zone.

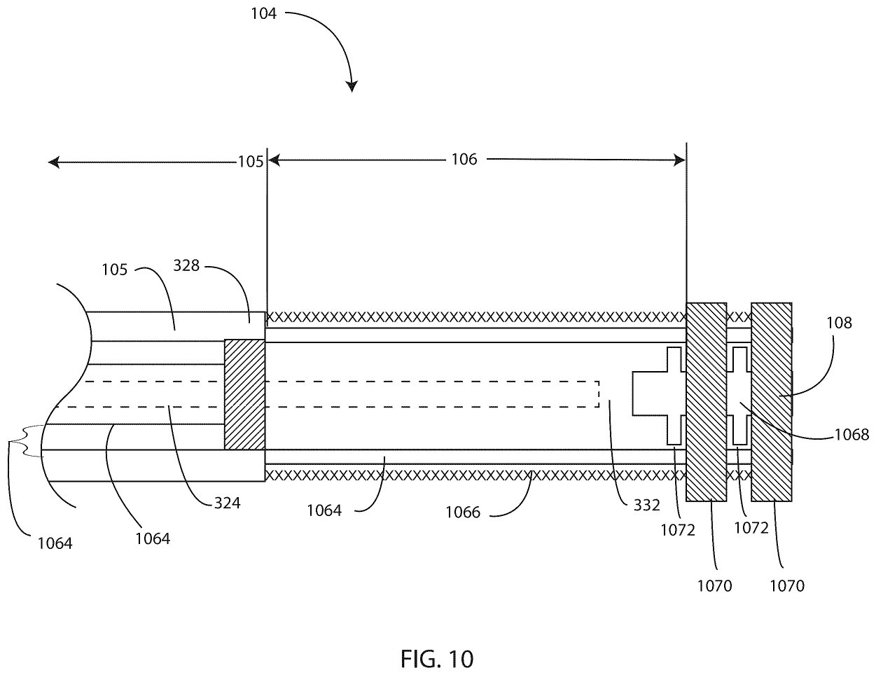

### Composite Shaft (FIG. 10)

Referring now to FIG. 10, a schematic view of a portion of a cryoablation shaft is shown in accordance with various embodiments herein. In various embodiments, the expansion chamber 106 of shaft 104 includes a supply tube 324, located concentrically within a composite return tube 1064 In various embodiments, the return tube can be a composite shaft. In various embodiments, the composite return tube 1064 is formed from one or more discrete layers. One possible return tube layer is a braid layer 1066 shown in FIG. 10.

In various embodiments, composite return tube 1064 can be formed from one or more polymer and/or braided layers as described in detail herein. After forming the discrete layers, the composite shaft may be heated to such a temperature that the discrete layers bond to each other forming a single composite layer. In some embodiments, some of the discrete layers may contribute to the radial strength of the expansion chamber and other discrete layers may contribute to the gas containment capability of the expansion chamber. In such embodiments, radial strength and gas containment may be achieved from a single composite layer. Such a configuration can enhance the tunability of the expansion chamber material properties while decreasing the diameter of the expansion chamber.

In some embodiments, an additional braid layer 1066 may be disposed over the remainder of the composite return tube 1064. The additional braid layer is selected to have braid properties that enhance the radial strength of the expansion chamber 106.

### Distal Fitting Examples (FIG. 10)

In various embodiments, the working fluid may be contained at the tip 108 of the shaft using a plug 1068 and one or more joints 1070. In various embodiments, the joints 1070 and/or the plug 1068 may be of a material or materials that are compatible to bond with the composite return tube 1064 and/or the braid layer 1066 to enhance the seal of the shaft 104. In various embodiments, the joints 1070 and/or the plug 1068 may be of a material or materials that are sufficiently strong as to enhance the burst strength of the expansion chamber.

In various embodiments, plug 1068 includes ridges 1072. The ridges 1072 improve the mechanical strength of the bonded joints 1070 by providing additional structural support to the bonds to resist the pressure within the expansion chamber.

### Leak Before Fail Seal/Engineered Seal

During a cryoablation procedure, some failure modes are less impactful than others. For example, a leak in the proximal end of the shaft presents much less risk to a patient than a leak near the tip of the shaft or a rupturing of the shaft. In various embodiments, the cryoablation system may be designed to detect a system failure quickly and automatically shut the system off.

In various embodiments, it is desirable to avoid blockages in the working fluid return path. Such blockages may result in the bursting of shaft 104, resulting in poor patient outcomes. In some embodiments, it may be desirable for the shaft to leak before it bursts. If the working fluid becomes blocked in the return path, the pressure in the return path will build until the eventual bursting of the shaft. For instance, in normal operating conditions, the operating pressure of the returning working fluid can range from about 150 psi to 200 psi, but in a blockage scenario, pressures can quickly reach values of greater than 1500 psi (e.g., about 1800 psi)

Referring back to FIG. 8, in various embodiments, the bond 847 between the reinforcing layer 844 and the slotted tube 830 may be designed to leak at a certain leak threshold pressure that is significantly lower than the burst pressure of the shaft 104. In some embodiments, the leak threshold pressure can be greater than or equal to 200 psi, 300 psi, 400 psi, or 500 psi. In some embodiments, the leak threshold pressure can be less than or equal to 1000 psi, 875 psi, 750 psi, 625 psi, or 500 psi. In some embodiments, the leak threshold pressure can fall within a range of 200 psi to 1000 psi, or 200 psi to 875 psi, or 300 psi to 750 psi, or 400 psi to 625 psi, or can be about 500 psi.

Upon the bond 847 leaking, the working fluid will breach from the reinforcing layer 844 into the space between the slotted tube and the insulating shaft. In various embodiments, the bond 847 is contained in the insulated portion 105 of the shaft 104. Consequentially, the leaked working fluid is contained in the vacuum circuit of the shaft between the slotted tube and the insulating shaft. In various embodiments, upon detecting the presence of working fluid in the vacuum circuit the cryoablation system is configured to shut down.

In various embodiments, seals designed to have a sufficiently low burst strength (e.g., a burst strength significantly lower than the burst strength of the shaft) may be placed at other locations of the shaft where leaking is preferable to bursting or other modes of failure. Generally speaking, leaks in the shaft near handle 102 are less impactful compared to leaks in the shaft closer to the tip 108. This is because leaks closer to the handle are quicker to detect and are less likely to penetrate the patient tissue. In various embodiments, the shaft may contain one, two, three, four, or more engineered seals placed along various locations of the shaft.

In various embodiments, the bond 848 between the first polymer layer 842 and the slotted tube 830 has a second leak threshold pressure which is significantly higher than the leak threshold pressure of bond 847. In some embodiments, the second leak threshold pressure can be greater than or equal to 1600 psi, 1750 psi, 1900 psi, 2050 psi, or 2200 psi. In some embodiments, the second leak threshold pressure can be less than or equal to 2800 psi, 2650 psi, 2500 psi, 2350 psi, or 2200 psi. In some embodiments, the second leak threshold pressure can fall within a range of 1600 psi to 2800 psi, or 1750 psi to 2650 psi, or 1900 psi to 2500 psi, or 2050 psi to 2350 psi, or can be about 2200 psi.

### Detection of Leak using Pressure Transducer

In various embodiments, the cryoablation system may be designed to detect a system failure quickly and automatically shut the system off. In various embodiments, the cryoablation system is configured to detect a leak in the first polymer layer 842 and/or in the reinforcing layer 844. In various embodiments, a vacuum is run in the insulated portion 105 of the shaft 104 between the slotted tube 830 and the insulating shaft 328. In the event where the working fluid leaks through the first polymer layer 842 or the reinforcing layer 844 (for instance at bonds 846 and 847), it breaches the vacuum zone between the slotted tube and the insulating shaft. Such an occurrence results in a rapid loss of vacuum pressure, which can be detected at a pressure transducer at the cryoablation system console (described in greater detail herein).

### Burst Valves Embodiments

In alternative embodiments, the cryoablation system may contain one or more burst valves in any of the shaft 104, the handle 102, and the console. For instance, the cryoablation system may contain one, two, three, four, or more burst valves at places throughout the length of the shaft and/or handle. The burst valves are configured to offer a flow obstruction that opens or bursts to vent when the pressure in the probe becomes dangerously high. In various embodiments, the one or more burst valves are replaceable after each shaft failure event.

The burst valves can be configured to rupture at a leak threshold pressure (which can be the same or different pressure than the leak threshold pressure at bond 847, as long as it is sufficiently below the burst strength of the shaft). The burst valves may be structured to send a signal to the console, or the console may detect a drop in the vacuum pressure of the vacuum circuit. The console may then automatically shut the flow of working gas off and the flow of pre-cooler gas off to avoid damage to the patient or system. Other suitable active or passive mechanical feature components can be used in lieu of burst valves.

In various embodiments, handle 102 may contain one or more burst valves (or other equivalent mechanical components). In various embodiments, the one or more burst valves are configured to leak in the working gas fluid circuit, the precooler fluid circuit, or both.

In a particular embodiment, the handle 102 or the insulated portion 105 of the flexible shaft may contain a T-chamber with a T-portion that extends radially from the return tube towards the insulating shaft, perpendicular to the axis of the return tube. At the ends of the T-portion, a burst valve is present. The burst valve can break to relieve pressure at a leak threshold pressure, such as one of the leak threshold pressures described herein, if an abnormal pressure builds up in the return tube.

### Laser Cut Windows

In various embodiments, the innermost layer of the return tube may contain one or more laser cut windows covered with a polymer layer. The polymer layer covering the laser-cut window can be engineered to leak at a leak threshold pressure, such as one of the leak threshold pressures described herein, if an abnormal pressure builds up in the return tube. For instance, laser cut windows can be cut in series in the return tube where working gas exhaust flows.

### Protective Lining

In various embodiments, certain portions of the shaft may include a protective lining. In some embodiments, the inner surface of the insulating shaft 328 can be coated with a protective lining. The protective lining may coat certain portions of the insulating shaft or the entire circumference and length of the insulating shaft. Additionally, or alternatively, the inner surface of the return tube 326 can be coated with a protective lining. The protective lining may coat certain portions of the return tube or the entire radius and length of the return tube. The protective lining can be formed from any of a metal lining, a sputter-coating, a sputter-coated metal lining, or the like. In various embodiments, the protective lining is configured to increase the burst strength of the return tube and/or insulating shaft and decrease the changes of a device failure. A thin coating may have minimal impact on flexibility.

### Diagnostic Tools to Measure Ice Ball Formation

In various embodiments, it is desirable to assess and monitor the formation of an ice ball over the duration of a cryoablation procedure. For instance, ice ball formation provides an indication of whether the shaft 104 (and in particular the expansion chamber 106) is correctly positioned relative to the patient tissue. In the event where the shaft is improperly positioned the ice ball may not form properly. Monitoring ice ball formation can also be useful to verify that the ice adequately covers the tissue and/or tumor with margin while protecting critical structures from the cryoablation. In addition, the ability to monitor the time at which the size of the ice ball stabilizes facilitates the application of an accurate and consistent ice ball residence time. Such benefits maximize the therapeutic value of the cryoablation procedure while minimizing complications.

In various embodiments, the cryoablation system 100 may include one or more diagnostic tools to measure ice ball size. In some embodiments, ice ball formation can be monitored using conventional medical imaging (e.g., computed tomography (or “CT), transcutaneous-ultrasonography (TUS), or MRI imaging depending on the application) to watch as freezing progresses within the tumor or tissue.

### Electrodes

In various embodiments, the cryoablation system 100 may include one or more electrodes to measure ice ball size. In some embodiments, ice ball size can be measured by receiving an impedance from at least one electrode along the shaft 104. In various embodiments, shaft 104 can include an electrode arrangement. The electrode arrangement includes at least one electrode, but may contain two, three, four, or more electrodes. The electrode arrangement may be disposed along any suitable location of the shaft, such as adjacent to the expansion chamber 106, or at the tip 108 of the shaft. Alternatively, the electrode arrangement may include a plurality of electrodes positioned along different portions of the shaft 104. For instance, electrodes can be placed along the shaft from the handle to the tip.

In various embodiments, the electrode arrangement is configured to engage the ice ball as the ice ball is formed over the expansion chamber during a cryoablation procedure. The progression of the ice ball formation results in a change in sensed impedance. In various embodiments, the cryoablation system may receive an impedance measurement from at least one electrode in the electrode arrangement. Based on the impedance measurement(s), one or more physical attributes of the ice ball can be determined. In various embodiments, the one or more physical attributes can include at least one of ice ball shape, ice ball size, and ice ball temperature. In a particular embodiment, the impedance measurement(s) can be used to track the growth of one more isotherms of the ice ball (as seen in FIG. 7). By tracking the evolving physical properties of the ice ball using the electrode assembly, the operator can assess the efficacy of the cryoablation treatment in real time and adjust (e.g., shaft position, cryoablation duration) when needed.

By positioning electrodes along the length of the flexible shaft and expansion chamber, the system can monitor the major axis of the ice ball.

In some embodiments, the electrode assembly (or another suitable heat application technique) can also be used to apply heat to the tissue around the expansion chamber 106 or tip 108. Such an application of heat reduces the likelihood of cell seeding upon device withdrawal of the shaft from the patient tissue.

### Thermocouple Devices

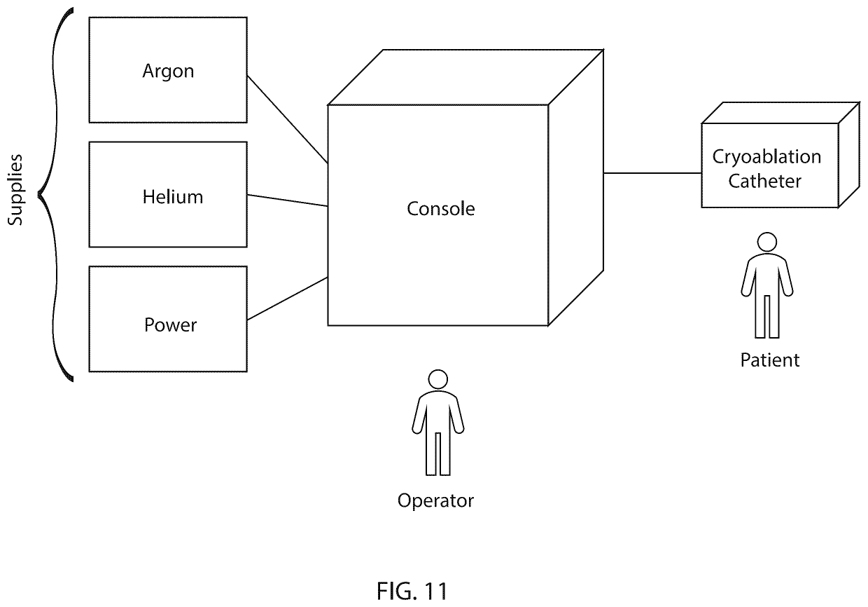

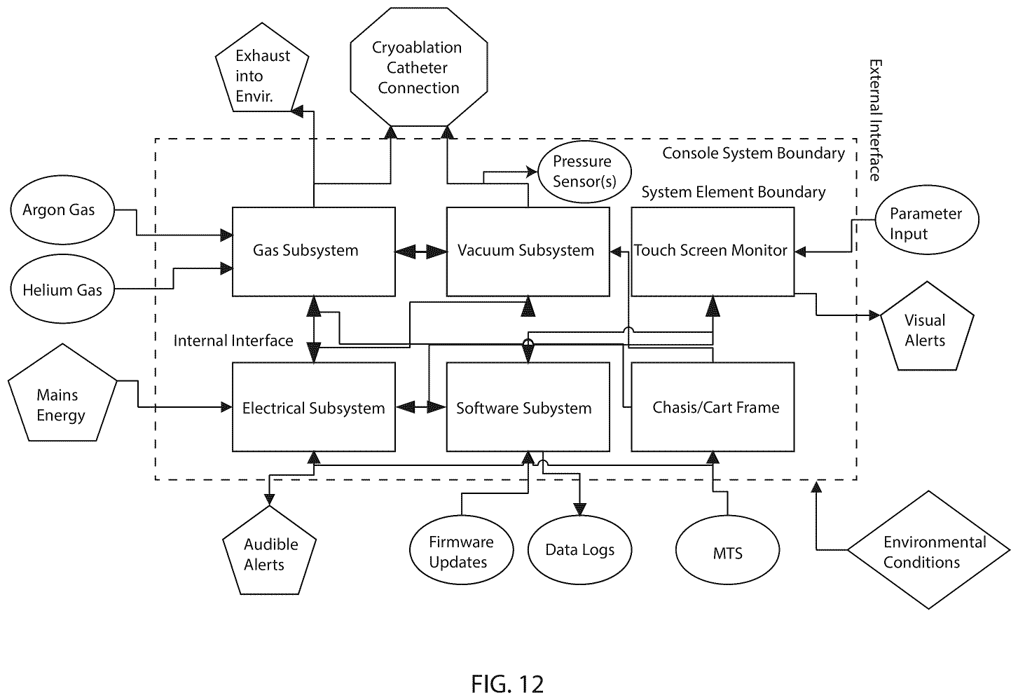

In various embodiments, the cryoablation system can include one or more thermocouple devices. For instance, the cryoablation system can include a thermocouple at the tip 108. The thermocouple may be soldered to the tip, glued, or otherwise adhered to the tip using a conductive epoxy, or joined to the tip by any other suitable means. The cryoablation system may include additional thermocouple devices along the length of the shaft 104 and/or in the handle 102. The thermocouple device may be linked to a temperature sensing component in the console (described in detail in FIGS. 11-12). The temperature sensing component may comprise numerous additional components modules such as resistors, diodes, and thermocouples.

One or more thermocouple devices disposed on or near the expansion chamber and/or tip of the shaft enables temperature measurements of the target nerve or tissue. Such a temperature measurements allow the operator to track the progress of the cryoablation procedure. In various embodiments, one or more thermocouple devices disposed in the handle allow the operator to observe the temperature of the pre-cooler and working fluids. Such a temperature measurement enables the operator to ensure that the precooler and working fluids are sufficiently cold prior to beginning a cryoablation procedure.

### Cryoablation System Overview (FIG. 11)

Referring now to FIG. 11, a schematic view of a cryoablation system is shown in accordance with various embodiments herein. The cryoablation system can include a console operatively connected to a cryoablation catheter and one or more supplies.

The first supply can be Argon or any other suitable cooling fluid (e.g., nitrogen, air, argon, krypton, xenon, N₂O, CO₂, CF₄) in embodiments. The argon supply is used to supply both the working fluid and the pre-cooler fluid to cryoablation catheter. In alternative embodiments, the system may contain separate fluid reservoir supplies or tanks for the working fluid and precooler circuits of the cryoablation catheter.

In various embodiments, the second supply can be for helium or any other heating fluid (e.g., hydrogen). In some embodiments, a heating fluid can be used as the working fluid. In such cases, the high-pressure stream of the working fluid may be at a pressure such that expansion via the Joule-Thomson orifice 332 may result in a temperature increase of the primary fluid, correspondingly resulting in heating of tissue surrounding the distal operating fluid. Such embodiments may be useful for thawing frozen tissue.

The third supply can be any suitable power supply configured to supply power to the console and the cryoablation catheter.

### Console (FIG. 12)

Referring now to FIG. 12, a schematic view of a console for a cryoablation system is shown in accordance with various embodiments herein. Elements of various embodiments of the console described herein are shown in FIG. 12. However, it will be appreciated that some embodiments can include additional elements beyond those shown in FIG. 12. In addition, some embodiments may lack some elements shown in FIG. 12. In various embodiments, the console may be in electrical and fluid communication with the cryoablation catheter, and may include one or more fluid reservoirs, coolant recovery reservoirs, valves, conduits, connectors, heaters, temperature sensors, pressure sensors, flow rate sensors, power sources, computing device(s), displays, and user input controls (e.g., keyboards, buttons). In various embodiments the console includes an internal interface and an external interface. The system boundary separates the internal interface from the external interface and provides connections to the console.

In various embodiments, the console includes electrical and software subsystems. The electrical and software subsystems include one or more computing devices. The computing device(s) may contain a microprocessor in communication with a memory via a bidirectional data bus. The memory can include read only memory (ROM) or random-access memory (RAM) for program storage and RAM for data storage. The electrical and software subsystems can be electrically connected to each of the other console subsystems to supply power to and transfer signals between the subsystems.

The electrical and software subsystems can also be electrically connected to the cryoablation catheter and can be configured to control the operating parameters of the cryoablation catheter. In an embodiment, the electrical and software subsystems may be programmable to control various system components, such as the one or more valves, to operate according to a duty cycle that includes opening and closing the one or more valves to regulate the flow of working through the cryoablation catheter.

In various embodiments, the software subsystem is configured to receive firmware updates and to output data logs regarding the status of the cryoablation system. In various embodiments, the electrical subsystem is configured to receive power from a power supply and to output audible alerts regarding the status of the cryoablation system.

In various embodiments, the console includes a gas to electrical subsystem. The gas to electrical subsystem can be in fluid communication with the argon and helium fluid supplies. Fluids may cycle to and from the supplies and the cryoablation catheter via the gas to electrical subsystem. Fluids may exhaust out of the cryoablation system into the ambient environment via the gas to electrical subsystem. Alternatively, the fluids may be recovered and reused (such as by pumping the recovered fluids back into their respective supplies). In such embodiments, the cryoablation system is effectively a closed loop system and fresh supplies of Argon and Helium are not required for each use. The gas to electrical subsystem can include one or more control gas solenoids to control the flow from the gas supplies. The gas to electrical subsystem can be configured to measure the pressures of the system fluids, drive and detect the freeze and/or thaw cycles in a cryoablation procedure and provide control for all the system fans (not shown).

In various embodiments, the console includes a vacuum subsystem. The vacuum subsystem can be in fluid communication with the internal gas interface. The vacuum subsystem is configured to provide vacuum to the cryoablation catheter via a vacuum connection. In various embodiments, the vacuum subsystem is electrically connected to the electrical subsystem to provide power to vacuum pump. The vacuum subsystem may also be in communication with one or more flow and/or pressure sensors to measure the vacuum pressure.

In various embodiments, the console includes a touchscreen monitor configured to provide information about the cryoablation system (e.g., operator instructions, status of the cryoablation procedure) and/or receive input from an operator (e.g., desired ablation time). The touch screen is configured to display information to a user via visual alerts and to receive feedback and/or commands from the operator. Operator feedback may be collected through any number of suitable means such as the touch screen, a keyboard, a microphone (to receive voice commands), or the like. In addition, or alternatively to the touchscreen monitor, the console may include any other suitable user interface such as one or more buttons, dials, indicator lights, or the like.

In various embodiments, the console includes a chassis and/or cart frame. In various embodiments the chassis and/or cart frame are configured to mount the touch screen monitor, vacuum subsystem, gas subsystem, and electrical subsystem to the console. In some embodiments, the cryoablation system is mobile and the cart frame includes wheels, or the like, to transport the system. In some embodiments, the cryoablation system may include one or more accessories (e.g., imaging systems, extra parts, or the like) that can be stacked on the chassis and/or cart frame.

### Redundant Safety Features

In various embodiments, the cryoablation system can include one or more sensors throughout the console and/or the cryoablation catheter (e.g., pressure sensors, temperature sensors, flow rate sensors) configured to detect a change in status of the gas flow or vacuum pressure, such as in the event of a leak in any portion of the cryoablation system. For instance, the console can include one or more multi-stage burst components (e.g., discs, valves, etc.) that are integrated into the console and in fluid communication with working fluid circuit and pre-cooler fluid circuit. The console is configured to detect a breach in any of the multi-stage burst components. Upon detecting a breach, the console may be configured to automatically shut down the cryoablation system to minimize damage to the system and/or patient, such as to close the supply of working gas, pre-cooler gas, vacuum pump, or more than one of these.

In various embodiments, the console is configured to control the extent of the cryoablation procedure (e.g., the size of the ice ball formed at the expansion chamber). For instance, the console is configured to gather feedback information from one or more sensors (e.g., electrodes, thermocouple devices) integrated into the cryoablation catheter. Based on the collected information from the one or more sensor, the console is configured to either maintain or alter the cryoablation operating parameters (e.g., cryoablation duration) to improve the outcome of the procedure.

In some embodiments, the console enables automated CT-scan integration. CT-scan integration can enable computer-aided catheter placement and/or ice ball size determination.

### Fluid Connector Lockout Configurations

In various embodiments the fluid connectors at the gas subsystem of the console are configured to ensure that the operator connects the correct supply gas. In an embodiment the fluid connectors at the gas subsystem can each include an electronic component. When the correct supply gas is connected to the gas subsystem the electrical component and/or one or more pressure transducers send a signal to the processor verifying that the connection is complete and correct. Alternatively, when the incorrect supply is connected to the gas subsystem (e.g., when the argon supply gas is connected to the helium gas connection), no signal is sent to the processor. In various embodiments, the console does not allow the cryoablation procedure to begin until the correct fluid connections are verified at the processor. In an alternative embodiment, physical measurement of each circuit of the gas subsystem is measured and then sent to the processor to verify that the connection is correct and complete.

In an alternative embodiment, each of the supply gas connectors at the gas subsystem can be of a different size and each of the gas supplies can be configured to only connect to the appropriately sized connector. In another embodiment, each of the supply gas connectors at the gas subsystem can contain a sniffer plumbed in-line with the gas connections. Each sniffer is configured to detect the type of gas being connected to its respective connector (e.g., Argon or Helium) and the cryoablation procedure can only begin if each sniffer detects the correct type of gas. Such connectors make it impossible for an operator to accidentally attach the incorrect gas supply.

### Redundant Safety Features-Procedures

### Toggle Vacuum Pump to Working Gas Circuit before Procedure

In various embodiments, the console is configured to execute one or more safety procedures to enhance the safety of the cryoablation procedure. For instance, the console may be configured to perform a vacuum check procedure to ensure the integrity of the probe before running a cryoablation procedure.

To execute the vacuum check procedure, during the process of steering the catheter to the desired position or after the cryoablation catheter is in position, the console is configured to toggle the vacuum pump from evacuating the insulation sleeve to instead evacuate the working gas channel. This is accomplished by closing the working gas exhaust opening, closing the working gas supply opening, pulling a vacuum on the working gas circuit, and checking for any change or deviation of the vacuum pressure (e.g., a decay in vacuum pressure) using a pressure transducer or a flow sensor. A steady vacuum pressure indicates that there are no leaks in the working gas channel. A change or deviation in vacuum pressure indicates that there are leaks in the working gas channel. Performing the vacuum check procedure ensures the airtightness of the flexible catheter after steering the flexible catheter to the treatment location and prior to cryoablation. This procedure reduces the risk of any working fluid leaking into the patient tissue or ambient environment.

The connection between the gas subsystem and the vacuum subsystem can be used to connect the vacuum pump to the working gas circuit to enable this safety procedure. In an alternate embodiment, the user can reconfigure the system to measure the working gas circuit against an isolated vacuum circuit.

### Step-Up Process for Connecting Gas Supply

After checking the seal of the working gas channel, the console can connect the working gas supply to the working fluid circuit. For instance, the operator may initiate the step-up procedure by interacting with the console (e.g., via the touch screen) and the gas circuit can switch the gas connections without operator intervention. Alternatively, an operator can manually connect the working gas supply to the working fluid circuit. Connecting the working gas supply to the working fluid circuit could potentially cause a leak, especially if very high-pressure gas is introduced into the system immediately. To mitigate the risk of a leak, the console is configured to use a step-up process. The console is first configured to introduce the working gas at a much lower pressure (about 600 psi or less) than the working pressure. The console or the operator is then configured to measure a pressure in the working gas channel, which will show a spike using a pressure transducer in the console for the initial introduction of gas. If there is no leak in the system, the pressure sensed at the pressure transducer will stabilize over time. After the pressure is stabilized, the console or the operator is then configured to gradually step up the gas pressure, until the working pressure (e.g., approximately 1800 psi) is reached. Each time the pressure is stepped up, the console or the operator waits for the gas pressure to stabilize. Identifying that a period of pressure stability has occurred before introducing a higher-pressure gas flow helps to ensure that there are no leaks in the system. The step-up procedure may be in place of or in conjunction with any of the other safety devices and procedures described herein.

The step-up procedure can also be used to introduce gas into the pre-cooler fluid circuit.

### Check of Vacuum Chamber Integrity

The console can be configured to check the integrity of the vacuum circuit before starting the cryoablation procedure in various embodiments. Upon starting up the vacuum pump, a vacuum is drawn into the insulated portion of the shaft. In various embodiments, it may take several minutes for the vacuum to reach the desired pressure and stability. In various embodiments, a pressure transducer is present somewhere in the vacuum circuit (either in the console or the cryoablation catheter). The pressure transducer can be electrically connected to the console. In various embodiments, the console is configured to measure the vacuum pressure in the vacuum circuit using the pressure transducer.

In one example, the console is configured to monitor the vacuum circuit pressure over a set period of time. During the set period of time, the console is configured to toggle off the vacuum. If the pressure increases at a higher rate than a threshold rate, the console detects a leak in the vacuum circuit. If the pressure holds or decays sufficiently slowly, then the console indicates that the vacuum is functioning properly. After running each of the safety checks, the console can allow for cryoablation to commence.

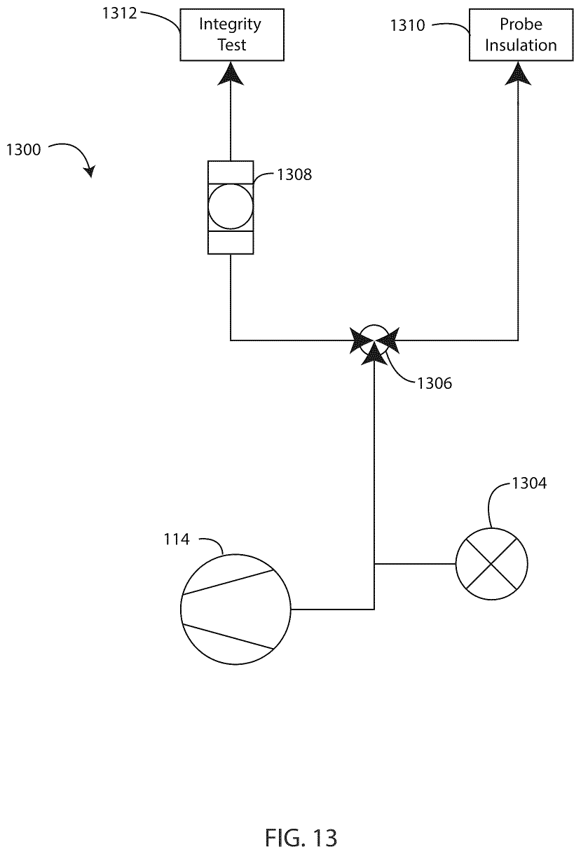

### Shaft Integrity Test Procedure (FIGS. 13-16)

In various embodiments, it is desirable to ensure that the cryoablation shaft is a closed system (devoid of leaks) prior to pumping the pressurized working and/or pre-cooling gasses through the shaft 104. It is undesirable to use positive pressure to test for leaks in a cryoablation system once the shaft has been inserted into the patient because, in the event of a leak, pressurized gas could flow through the system and into the patient.

To avoid such risks, a shaft integrity test can be implemented prior to flowing the pressurized working and pre-cooling gases through the shaft 104. In various embodiments, the shaft integrity test utilizes the ability of the shaft to achieve and maintain vacuum between the supply tube 324 and the return tube 326 to determine that the probe is a closed system. If a sufficiently low level of vacuum cannot be reached and maintained by the shaft, a determination that the shaft has been compromised will be made. In the event that the shaft is inside of a patient during the shaft integrity test and a leak is present, fluid from the patient may flow into the shaft. Although such a scenario could damage the cryoablation system, harm to the patient is unlikely. The systems and methods for performing the shaft integrity test are described in further detail herein.

### Testing Apparatus (FIG. 13)

Referring now to FIG. 13, a schematic view of a testing apparatus for performing the shaft integrity test is shown in accordance with various embodiments herein. To execute the shaft integrity test, the cryoablation system is configured to toggle a vacuum source 114 between evacuating the vacuum circuit (e.g., the space between the return tube 326 and the insulating shaft 328) and evacuating the working gas return path (e.g., the space between the supply tube 324 and the return tube 326). In some embodiments, the testing apparatus is part of the console shown and described in FIG. 12.

In various embodiments, the testing apparatus 1300 can include a vacuum source 114. The vacuum source can be a vacuum pump, or the like. In some embodiments, the vacuum source 114 is an active vacuum. For instance, the vacuum source 114 may be a vacuum pump, or the like. In normal operation, the vacuum source 114 is in operative communication with the vacuum chamber. However, while executing the shaft integrity test, the vacuum source is toggled into operative communication with the working gas circuit. In alternate configurations, a cryoablation system 100 can include a first vacuum source for evacuating the vacuum circuit and a second vacuum source for evacuating the working return path.

The vacuum source can be brought into communication with any portion of the working gas circuit to perform the shaft integrity test. In various embodiments, the vacuum source is toggled into communication with the gas return path. In various embodiments, the vacuum source is toggled into communication with the supply tube. The gas return path and the supply tube are components of the working gas circuit and are in fluid communication with each other.

In various embodiments, the testing apparatus 1300 can include a vacuum pressure sensor 1304. In various embodiments, the pressure sensor is configured to monitor the pressure of the working gas return path and/or the vacuum circuit. The pressure sensor 1304 can be any suitable type of pressure sensor, such as a gauge pressure sensor, or the like. In some embodiments, the testing apparatus can include two, three, or more pressure sensors configured to monitor the pressure at various locations or positions within the cryoablation system.

In various embodiments, the testing apparatus 1300 can include a valve 1306. The valve 1306 can be a three-way valve. In various embodiments, the valve 1306 can be actuated to toggle between a first position connecting the vacuum source to the vacuum chamber and a second position connecting the vacuum source to the working gas circuit. In various embodiment, the testing apparatus 1300 can further include a user interface (such as the touch screen monitor disposed on the console) such that the cryoablation system operator can selectively actuate the valve 1306 to toggle between the first position and the second position via the user interface.

In various embodiments, the testing apparatus 1300 can include a probe insulation manifold 1310. In various embodiments, the probe insulation manifold 1310 can include one or more mating features (e.g., O-rings) and is configured to sealingly connect to the cryoablation probe. In various embodiments, when the cryoablation probe is connected to probe insulation manifold 1310, the vacuum chamber is in fluid communication with the vacuum source 114. Accordingly, when the vacuum source 114 is powered on and the valve 1306 is toggled to its first position, the vacuum source is configured to evacuate the vacuum chamber, providing insulation to the cryoablation shaft 104.

In various embodiments, the testing apparatus 1300 can include an integrity test manifold 1312. In various embodiments, the integrity test manifold 1312 can include one or more mating features (e.g., O-rings) and is configured to sealingly connect to the cryoablation probe. In various embodiments, a cryoablation shaft can be disconnected from the probe insulation manifold 1310 and connected to the integrity test manifold 1312, or vice versa. In various embodiments, when the cryoablation probe is connected to probe insulation manifold 1310, the vacuum chamber is in fluid communication with the working gas return path. Accordingly, when the vacuum source 114 is powered on and the valve 1306 is toggled to its second position, the vacuum source is configured to evacuate the working gas return path, such as when performing the shaft integrity test.

In various embodiments, the testing apparatus 1300 can include a moisture trap 1308. A moisture trap, as defined herein, is any device configured to prevent undesired moisture from entering a system. The moisture trap 1308 can be any suitable type of moisture trap, such as a sump, or the like. In various embodiments, the moisture trap is positioned within the testing apparatus 1300 between the integrity test manifold 1312 and the vacuum source 114. The human anatomy is filled with fluids and running the shaft integrity test on a compromised shaft could result in bodily fluids being pulled into the shaft 104. Such fluids can be pulled into the console via the vacuum source 114 and damage the internal components of the cryoablation system. Accordingly, placing a moisture trap upstream of components prone to damage (e.g., the vacuum source 114 and pressure sensor(s) 1304) can prevent such components from being destroyed while performing the shaft integrity test on a compromised shaft.

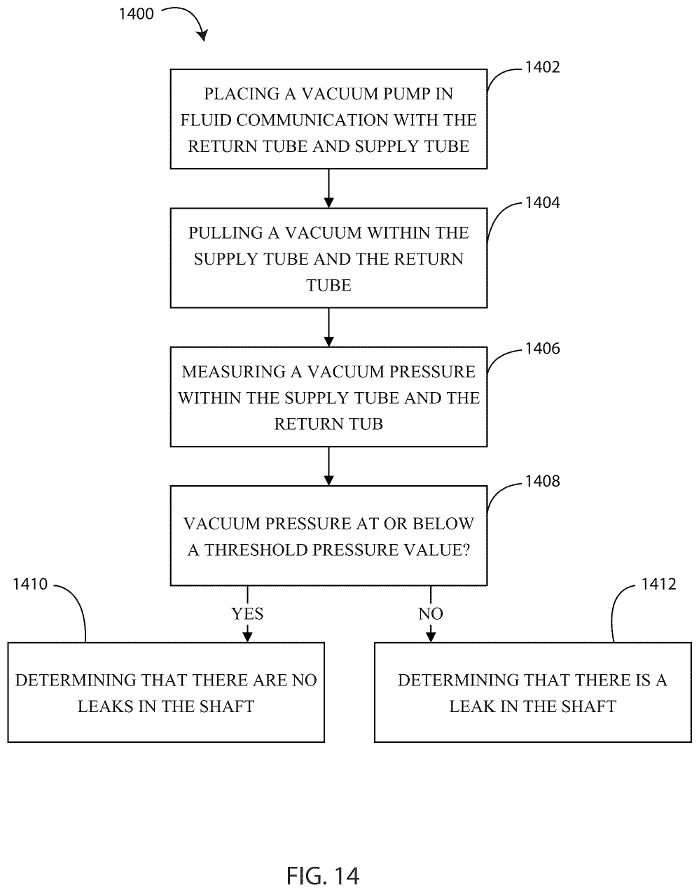

### Shaft Integrity Test Method (FIGS. 14-16)

Many different methods are contemplated herein, including, but not limited to, methods of making a cryoablation system, making a testing system for a cryoablation system, methods of testing a cryoablation system, methods of using a cryoablation system, methods of using a cryoablation testing system, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

Referring now to FIG. 14, a method of performing a shaft integrity test is shown in accordance with various embodiments herein. In some embodiments, the method 1400 can be performed after navigating a cryoablation shaft 104 to a cryoablation site within a patient, but before pumping cryogenic gases into the shaft to perform a cryoablation procedure. In some embodiments, the method 1400 can be performed prior to navigating the cryoablation shaft 104 to the cryoablation site. In various embodiments, a cryoablation procedure can include multiple ablations. In some embodiments, the method 1400 can be performed after a first ablation has been performed in a patient, but prior to performing a second ablation at the same or in a different location. In some embodiments, the method 1400 can be performed after a cryoablation procedure has been performed in a patient, but prior to performing an ablation. In some embodiments, the method 1400 can be performed after an ablation has been performed in a patient at a location and the cryoablation device has been navigated to a subsequent location, but prior to performing a subsequent cryoablation procedure.

In various embodiments, the method is performed using a cryoablation system 100. The cryoablation system can include a shaft 104 having a supply tube 324, a return tube 326, and an insulating shaft 328. The shaft can include an expansion chamber 106 towards a distal end of the shaft, wherein a working fluid is configured to travel from a proximal end to the distal end of the shaft, expand in the expansion chamber, and travel back to the proximal end of the shaft between the supply tube 324 and the return tube 326 (also referred to as the working gas return path herein).

In various embodiments, the method 1400 can include the step 1402 of placing a vacuum source 114 in fluid communication with the return tube 326 and supply tube 324. If the cryoablation probe is connected to probe insulation manifold 1310, step 1402 can include disconnecting the cryoablation probe from the probe insulation manifold 1310 and connecting the cryoablation probe to the integrity test manifold 1312. If the valve 1306 is in its first position, step 1402 can further include toggling the valve to its second position and placing the vacuum source 114 in fluid communication with the return tube 326 and supply tube 324.

In various embodiments, the method 1400 can include verifying that the working fluid source 110 is switched off such that no working gas is being pumped into the shaft 104. In various embodiments, the method 1400 can include verifying that the pre-cooler fluid source 112 is switched off such that no pre-cooler fluid is being pumped into the shaft 104.

In various embodiments, the method 1400 can include the step 1404 of pulling a vacuum within the supply tube 324 and return tube 326. In various embodiments, pulling a vacuum includes powering the vacuum source 114 on and placing it in fluid communication with the space between the supply tube 324 and return tube 326 such that the vacuum source is configured to evacuate the working gas return path. In various embodiments, pulling a vacuum within the supply tube 324 and return tube 326 evacuates any residual fluid (e.g., air, working gas) from the working gas return path.

In various embodiments, the vacuum source 114 is powered on and running prior to connecting the vacuum source 114 to the working gas return path via the integrity test manifold 1312. The vacuum source 114 can be set to run until it reaches a steady state prior placing it in fluid communication with the supply tube 324 and return tube 326. Once a steady state has been reached, valve 1306 can be opened fluidly connecting the vacuum source 114 to the shaft 104.

In various embodiments, the vacuum is pulled on the working fluid return path for a predetermined amount of time. In some embodiments, the predetermined amount of time can be greater than or equal to 5, 10, 15, 20, or 25 seconds. In some embodiments, the predetermined amount of time can be less than or equal to 100, 80, 65, 45, or 25 seconds. In some embodiments, the predetermined amount of time can fall within a range of 5 to 100 seconds, or 10 to 80 seconds, or 15 to 65 seconds, or 20 to 45 seconds, or can be about 25 seconds.

In various embodiments, the method 1400 can include the step 1406 of after pulling the vacuum for the predetermined amount of time, measuring a vacuum pressure within the supply tube 324 and the return tube 326. In some embodiments, waiting for at least the predetermined amount of time allows for the vacuum pressure in the shaft to reach a steady state. In various embodiments, the vacuum pressure is measured with pressure sensor 1304.

In various embodiments, step 1408 can include comparing the measured vacuum pressure to a threshold pressure value. In some embodiments, the threshold pressure value can be greater than or equal to 0.00 Torr, 0.02 Torr, 0.03 Torr, or 0.05 Torr. In some embodiments, the threshold pressure value can be less than or equal to 0.10 Torr, 0.08 Torr, 0.07 Torr, or 0.05 Torr. In some embodiments, the threshold pressure value can fall within a range of 0.00 Torr to 0.10 Torr, or 0.02 Torr to 0.08 Torr, or 0.03 Torr to 0.07 Torr, or can be about 0.05 Torr. The exact threshold pressure value can vary based on the configuration of the cryoablation system, but for exemplary purposes, the threshold pressure value will be set to 0.05 Torr.

In various embodiments, the method 1400 can include the step 1410 of determining that there are no leaks in the shaft if the vacuum pressure is at or below a threshold pressure value. In various embodiments, having the vacuum pressure in the space between the supply tube 324 and return tube 326 stabilize to at or below the threshold pressure value is indicative that the shaft is capable of holding a vacuum. If the shaft is able to maintain a sufficiently low vacuum pressure, it is highly likely that the shaft is devoid of leaks.

In various embodiments, the method 1400 can include the step 1412 of determining that there is a leak in the shaft if the vacuum pressure is above the threshold pressure value. In various embodiments, a vacuum pressure above the threshold pressure value is indicative that the shaft is not capable of holding a sufficient vacuum. Not being able to hold a sufficiently low vacuum is indicative of a compromised shaft.

Upon determining that there is a leak in the shaft, an indication can be recorded in a memory system of the console that a leak is present in the particular shaft. In various embodiments, the console stores a log of cryoablation probes and test results, such as fail or pass and a date and time of the test, associated with each probes. Further, an indication can be recorded that a particular shaft cannot be used in a cryoablation procedure after a leak has been detected. In various embodiments, the method 1400 can include upon determining that there is a leak in the shaft, automatically restricting the working and/or precooler gasses from being pumped into the cryoablation shaft 104.

In various embodiments, the method 1400 can include the step of recording a leak indication upon determining that there is a leak in the shaft. In some embodiments, the leak indication can include one or more visual and/or audible alerts to the system operator. In some embodiments, the leak indication can include instructions to the operator to perform any of not connecting the working and/or pre-cooler gas supplies to the compromised shaft, removing the compromised shaft from the patient, disposing of the compromised shaft, or the like.

In various embodiments, the method 1400 can include the step of storing the leak indication on the cryoablation probe. The leak indication can be stored electronically in a storage device located in the device, in some embodiments. For example, the device can include a data chip, such as an electronically erasable programmable read-only memory (EEPROM). In some embodiments, a data chip is housed in the handle 102. Upon connecting a cryoablation probe with a leak indication to the console, the console software can read the leak indication from the probe and alert the system operator of a faulty shaft and/or restrict cryogenic gases from being pumped into the shaft.

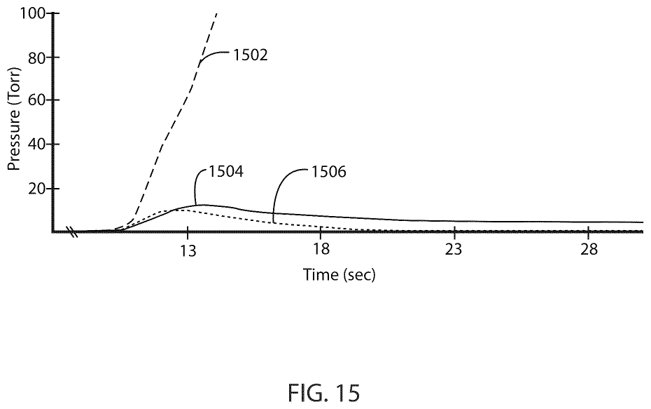

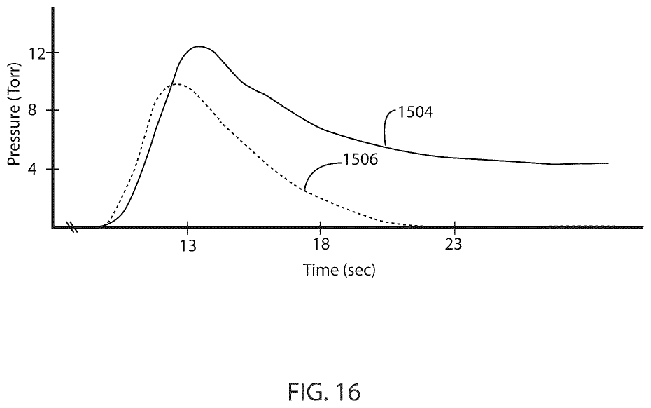

In various embodiments, the method 1400 can include, upon pulling the vacuum within the supply tube and the return tube, continuously monitoring the vacuum pressure over time during the predetermined time. In various embodiments, the cryoablation system is configured to collect data regarding shaft pressure vs. time such as seen in FIGS. 15-16.

Referring now to FIG. 15, an exemplary plot of shaft pressure vs. time for three different shafts is shown in accordance with various embodiments herein. Plot 1502 corresponds to the vacuum pressure for a first shaft, plot 1504 corresponds to the vacuum pressure for a second shaft, and plot 1506 corresponds to the vacuum pressure for a third shaft.

In various embodiments, the method 1400 can include determining that the shaft is majorly compromised upon measuring a continuous increase in the vacuum pressure towards ambient pressure. A majorly compromised shaft is unable to hold a vacuum of any kind and the pressure in the shaft will ultimately equalize to the pressure of its surrounding environment. A shaft can be majorly compromised when it is not closed at its distal end (e.g., the distal operating tip 108 has become dislodged) or when the shaft has a substantial leak or rupture. In the example of FIG. 15, plot 1502 corresponds to pressure data for a majorly compromised shaft. It can be seen that the shaft pressure increases rapidly towards ambient pressure and no vacuum is held.

Referring now to FIG. 16, an exemplary plot of shaft pressure vs. time for two different shafts is shown in accordance with various embodiments herein. FIG. 16 is a zoomed in version of FIG. 15 with plot 1502 removed for clarity.

In various embodiments, the method 1400 can include monitoring an initial rise in the vacuum pressure and after the initial rise in vacuum pressure monitoring that the vacuum pressure has stabilized to a value that is above the threshold pressure value. The method 1400 can further include determining that there is a leak in the shaft based on the stabilized pressure being above the pressure threshold value. A leaking shaft can sometimes maintain a partial vacuum, but due to the presence of the leak(s), the vacuum pressure will not equalize to the vacuum threshold pressure.

In the example of FIGS. 15-16, plot 1504 corresponds to pressure data for a leaking shaft. In the example of FIGS. 15-16, the vacuum source 114 is running at a steady state prior to opening the valve 1306 and connecting the vacuum source to the shaft 104. Accordingly, the vacuum pressure will be approximately zero until the valve 1306 is opened, fluidly connecting the vacuum source 114 to the shaft 104. The pressure sensor will detect an initial rise in pressure as the vacuum source 114 evacuates any fluids (e.g., air, working gas) from the shaft 104 and any connecting tubing. In the example of FIGS. 15-16 the pressure value then peaks at approximately 13 seconds. The exact peak of the pressure spike is defined by the volume of the shaft and of any tubing between the pressure sensor 1304 and the integrity test manifold 1312.

After reaching a peak vacuum pressure at approximately 14 seconds, plot 1504 stabilizes (as time advances) to a value of approximately 4 Torr. As the vacuum pressure has stabilized to a value (4 Torr) that is above the threshold pressure value (0.05 Torr), it is determined that the shaft is compromised (has one or more leaks).

In various embodiments, the method 1400 can include, monitoring an initial rise in the vacuum pressure and after the initial rise in vacuum pressure, monitoring that the vacuum pressure has stabilized to a value that is at or below the threshold pressure value. The method can further include determining that there are no leaks in the shaft based on the stabilized pressure value being at or below the threshold pressure value.

In the example of FIGS. 15-16, plot 1506 corresponds to pressure data for a closed shaft (no leaks). After reaching a peek vacuum at approximately 12 seconds, plot 1506 stabilizes (as time advances) to a value of approximately 0.01 Torr. As the vacuum pressure has stabilized to a value (0.01 Torr) that is below the threshold pressure value (0.05 Torr), it is determined that the shaft is not compromised (has no leaks).

### Evaluation of Slope of Initial Vacuum Pressure

As can be observed from FIGS. 15-16, plots 1502, 1504 and 1506 have different rates of change or slope of the pressure value. The rate of change of these plots can be used to identify probes with open distal ends or with leaks. In various embodiments, the method 1400 can include monitoring an initial rise in the vacuum pressure and measuring a rate of change in the initial rise in the vacuum pressure. If the rate of change is above a first threshold rate of change, the method can include determining that there is a leak in the shaft.

For example, plot 1502 in FIG. 15 rises very quickly from 0 Torr to about 60 Torr within about 5 seconds, which is a rate of change is about 12 Torr/second. In some embodiments, the threshold rate of change can be greater than or equal to 5 Torr/second (0.7 kPa/second), 8 Torr/second (1.1 kPa/second), 11 Torr/second (1.5 kPa/second), 14 Torr/second (1.9 kPa/second), 17 Torr/second (2.3 kPa/second), or 20 Torr/second (2.7 kPa/second), or can be an amount falling within a range between any of the foregoing.

### Markers for Imaging Systems

The joint 334 (FIG. 3) and the distal tip 108 (FIGS. 8 and 10) are configured to appear on imaging systems. The joint 334 can include a marker band of radiopaque materials, such as tungsten or platinum iridium or other appropriate materials. The material and dimensions of the distal tip 108 can be configured to appear on imaging systems. Examples of materials for the distal tip 108 include stainless steel and nitinol. The markers at either end of the active zone of the device assist the physician with visually identifying the location of the active zone of cryoablation with respect to the patient's anatomy and the target zone for cryoablation. Examples of imaging systems include ultrasound, fluoroscopy, cone beam CT, and MRI systems.

### Introduction Options

In some embodiments, the catheter system is delivered using a sheath introduction system. An example of a sheath introduction system is a steerable sheath. Alternatively, the catheter system can be steerable. In another embodiment, the catheter system includes a monorail lumen along a portion of the catheter to facilitate introduction.

The concepts described herein can be applied in the context of and used in connection with cryoablation systems and components described in the following four U.S. nonprovisional patent applications, which are filed on the even date herewith, which are incorporated by reference herein in their entireties: U.S. Nonprovisional patent application Ser. No. ______, titled “Cryoablation Catheter Shaft Construction,” having attorney docket number 115.0421USU1; U.S. Nonprovisional Patent Application No. ______, titled “Multiple Gas Circuit Connector and Method for Cryoablation System,” having attorney docket number 115.0423USU1; U.S. Nonprovisional patent application Ser. No. ______, titled “Delivery Systems for Cryoablation Device,” having attorney docket number 115.0424USU1; and U.S. Nonprovisional patent application No. ______, titled “Distal Tip Structure for Cryoablation Probe,” having attorney docket number 115.0438US01.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method for detecting leaks in a shaft of a cryoablation system, the shaft comprising a supply tube, a return tube, and an expansion chamber towards a distal end of the shaft, the shaft configured to allow a working fluid to travel from a proximal end to the distal end of the shaft, expand in the expansion chamber, and travel back to the proximal end of the shaft between the supply tube and the return tube, the method comprising;
placing a vacuum pump in fluid communication with the return tube and supply tube;
pulling a vacuum within the supply tube and the return tube;
after pulling the vacuum for a predetermined amount of time, measuring a vacuum pressure within the supply tube and the return tube;
if the vacuum pressure is at or below a threshold pressure value, determining that there are no leaks in the shaft; and
if the vacuum pressure is above the threshold pressure value, determining that there is a leak in the shaft.

2. The method of claim 1, further comprising upon determining that there is a leak in the shaft, recording a leak indication.

3. The method of claim 1, wherein the threshold pressure is approximately 0.05 Torr (6.67 Pa).

4. The method of claim 1, wherein the predetermined amount of time is between about 10 seconds and about 45 seconds.

5. The method of claim 1, further comprising, upon pulling the vacuum within the supply tube and the return tube, continuously monitoring the vacuum pressure over time during the predetermined time.

6. The method of claim 5, further comprising, upon measuring a continuous increase in the vacuum pressure towards ambient pressure, determining that the shaft is not closed at the distal end.

7. The method of claim 5, further comprising:
monitoring an initial rise in the vacuum pressure;
after the initial rise in vacuum pressure, monitoring that the vacuum pressure has stabilized to a value that is above the threshold pressure value; and
determining that there is a leak in the shaft.

8. The method of claim 5, further comprising:
monitoring an initial rise in the vacuum pressure;
after the initial rise in vacuum pressure, monitoring that the vacuum pressure has stabilized to a value that is at or below the threshold pressure value; and
determining that there are no leaks in the shaft.

9. The method of claim 5, further comprising:
monitoring an initial rise in the vacuum pressure;
measuring a rate of change in the initial rise in the vacuum pressure; and
if the rate of change is above a threshold rate, determining that the shaft is compromised.

10. The method of claim 1, wherein the method is performed before the cryoablation system is introduced into a patient.

11. The method of claim 1, wherein the method is performed after the cryoablation system is introduced into a patient, during or after steering the cryoablation system to a treatment site.

12. The method of claim 1, wherein the method is performed after a first cryoablation procedure has been completed with the cryoablation system and before a second cryoablation procedure is to be performed with the cryoablation system.

13. A cryoablation system comprising:
a working gas circuit;
a vacuum chamber;
a vacuum pump configured to pull a vacuum on one of the working gas circuit and the vacuum chamber;
a console configured to toggle the vacuum pump between the working gas circuit and the vacuum chamber; and
a pressure sensor configured to measure a vacuum pressure in the working gas circuit;
wherein the console is further configured to:
toggle the vacuum pump to the working gas circuit;
control the vacuum pump to pull a vacuum on the working gas circuit;
after the vacuum has been pulled for a predetermined amount of time, measure a vacuum pressure in the working gas circuit with the pressure sensor; and
if the vacuum pressure is above a threshold pressure value, determining that a shaft is compromised.

14. The cryoablation system of claim 13, wherein the console is further configured to pull the vacuum within the working gas circuit, and continuously monitor the vacuum pressure over time during the predetermined time.

15. The cryoablation system of claim 14, wherein the console is further configured to measure a continuous increase in the vacuum pressure towards ambient pressure and determine that the shaft is not closed at a distal end.

16. The cryoablation system of claim 14, wherein the console is further configured to:
monitor an initial rise in the vacuum pressure;
after the initial rise in vacuum pressure, monitor that the vacuum pressure has stabilized to a value that is above the threshold pressure value; and
determine that there is a leak in the shaft.

17. A cryoablation system comprising:
a pre-cooler fluid circuit;
a working fluid circuit;
a vacuum circuit; and
a shaft, the shaft comprising
an insulated portion, wherein the vacuum circuit is defined within the insulated portion; and
an expansion chamber, the expansion chamber comprising:
a supply tube having a distal outlet in the expansion chamber, wherein fluid from the working fluid circuit travels through the supply tube and expands in the expansion chamber;
a first layer, wherein the first layer is configured to contain fluid from the working fluid circuit; and
a second layer, wherein the second layer is configured to increase a radial strength of the expansion chamber;
wherein the shaft has a lower burst strength in the insulated portion at a first location and a higher burst strength in the expansion chamber at a second location.

18. The cryoablation system of claim 17, wherein the shaft comprises an inner metal tube and the first layer is sealed to the inner metal tube at a distal end of the inner metal tube, where the first location is a seal location between the first layer and the inner metal tube.

19. The cryoablation system of claim 17, the first layer comprising PET and the second layer comprising a polymer or
a braided material.

20. The cryoablation system of claim 17, further comprising a burst valve at the first location.
